(19) United States
(12) Patent Application Publication
(10) Pub. No.: US 2024/0238410 A1
(43) Pub. Date: Jul. 18, 2024

(54) SAFER VACCINES

(71) Applicant: B & H Biotechnologies, LLC, Chicago, IL (US)

(72) Inventor: Huiru Wang, Hangzhou, Zhejiang (CN)

(21) Appl. No.: 18/561,617

(22) PCT Filed: May 21, 2021
(86) PCT No.: PCT/CN2021/095146

§ 371 Date: Nov. 16, 2023

(51) Int. Cl. A61K 39/215; A61K 39/00; A61K 39/145; A61P 37/04; C07K 16/10; C12N 7/00; G01N 33/569

(52) U.S. Cl. CPC A61K 39/215; A61K 39/145; A61P 37/04; C07K 16/1003; C07K 16/1018; C12N 7/00; G01N 33/56983; A61K 2039/53; C12N 2760/16034; C12N 2770/18033; G01N 2333/11; G01N 2333/165; G01N 2469/20

(57) ABSTRACT

The invention provides safer vaccines that induce less adverse reactions particular the serious adverse reactions in a host. Also provided are compositions including these safer vaccines, as well as polynucleotides, vectors, host cells, methods, and kits related thereto. Further provided are methods and kits for preventing or treating infectious diseases, infection-relating diseases, and adverse reactions of vaccines in an individual by administering to the individual a safer vaccine that induce less adverse reactions, or by administering to the individual a pathogenic antigen that neutralize pathogenic antibodies. Yet further provided are methods for identification of the presence of pathogenic antibodies inducible by a pathogen or the vaccines relating to the pathogen.

### FIELD

This invention relates to safer vaccines comprising non-pathogenic vaccine antigens of a pathogen that induce less adverse reactions in a host. The safer vaccines are preferably mRNA, DNA, recombinant, or subunit vaccines, as well as compositions, polynucleotides, vectors, host cells, methods of production, methods of use, and kits related thereto.

### BACKGROUND

Vaccines are the most effective approach to prevent infectious diseases. However, vaccines are not perfect as they may cause serious adverse reactions even death. For example, the swine influenza vaccine in 1976 might be related to about 500 cases of Guillain-Barre syndrome (GBS) and 25 deaths that the vaccine had to be called off (US CDC, VAERS). The 2009 monovalent H1N1 (swine) influenza vaccine might have induced 636 serious health events, including 103 cases of GBS and 51 deaths in the United States (US CDC, VAERS). The vaccination of the mRNA vaccine of the Coronavirus Disease 2019 (COVID-19) virus, the SARS-CoV-2 might have induced 2,509 deaths in the United States among people who received a COVID-19 vaccine (0.0017%) during Dec. 14, 2020, through Mar. 29, 2021 (US CDC, VAERS). The adenoviral vector based COVID-19 vaccine (AZD1222) might have induced thrombotic response (J Thromb Haemost. 2021 Apr. 20. doi: 10.1111/jth.15347). Thus far, the pathogenic mechanisms of the serious adverse reactions of vaccines including COVID-19 and influenza vaccines remained unclear. Neither there are no any medicines for preventing and treating the serious adverse reactions of vaccines due to the unclear pathogenic mechanisms.

Therefore, there remains a need for effective and safer vaccines that induce less adverse reactions for a better control of infectious diseases particularly for a better control of a pandemic caused by highly pathogenic viruses (e.g. COVID-19 pandemic).

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

To meet the demand for better vaccines, disclosed herein are safer mRNA, or DNA, recombinant vaccines, or subunit vaccines, that comprise at least one of the non-pathogenic antigens of a pathogen, as well as compositions, polynucleotides, vectors, host cells, methods of production, and kits related thereto. Further disclosed are methods of preventing or treating infectious diseases, infection-relating disease, and adverse reactions of vaccines in an individual by administering safer vaccines that induce at least one of the non-pathogenic antibodies to the host.

These safer vaccines and methods are based in part on the unexpected discovery that certain antibodies specific for the spike protein (S1) of the COVID-19 (SARS-CoV-2) virus, or specific for the spike glycoprotein of the SARS-CoV virus (SARS-CoV S) can bind in vivo to fetal tissues or diseased (e.g. inflammatory) tissues of a host, activating self-attack immune responses and inducing systematic inflammation and damages of multiple organs including lung, kidney, hart brain, liver and intestine. Moreover, the pathogenic antibodies of anti-COVID-19 (SARS-CoV-2) S1 or anti-SARS-CoV S antibody caused postpartum labor, still birth of pregnant females, and neonatal death and neonatal sudden death of pregnant females. Similar or severer results were also observed with two of human monoclonal antibodies isolated from the patients infected with the COVID-19 (SARS-CoV-2) virus. The two monoclonal antibodies are specific for the receptor binding domain (RBD) of the spike protein (S-RBD) of the COVID-19 virus (SARS-CoV-2). The in vivo results demonstrate for the first time that certain anti-COVID-19 S antibodies are pathogenic. The pathogenic antibodies may be responsible for the serious adverse reactions of COVID-19 vaccines. The data also suggested that mRNA, DNA, recombinant, or subunit COVID-19 vaccines designed to target spike protein of SARS-CoV-2 virus can induce severer adverse reactions since those vaccines induce higher levels of anti-COVID-19 S antibodies including the pathogenic antibodies. It should be noted that the majority (70% or more) of the anti-COVID-19 S antibodies are safe since the pathogenic antibodies only take less than 30%.

Additionally, the present application describes the surprising finding that if the pathogenic anti-COVID-19 S1 antibody was mixed and administrated with equal amount of the non-pathogenic antibody of anti-COVID-19 nucleocapsid (N) protein, the sick and death rate of the pathogenic antibody was significantly decreased compared to the controls treated with the anti-COVID-19 S1 antibody alone (p value: 0.01, Table 1). Moreover, the sick and death rate induced by one of the pathogenic monoclonal antibodies specific for the S-RBD of the COVID-19 virus, was also significantly decreased when a mixture of the antibody with other two non-pathogenic monoclonal antibodies specific for the S-RBD of the COVID-19 virus was administrated (p value: 0.04, Table 1). The pathogenic and non-pathogenic monoclonal antibodies were isolated from patients with the COVID-19 infection (Hansen et al., Science 369, 1010-1014; 2020). The results suggest that co-existing of non-pathogenic antibodies can reduce the pathogenicity of pathogenic antibodies. In another word, a vaccine capable of inducing non-pathogenic antibodies are safer.

Therefore, in one aspect, provided herein are safer vaccines comprising at least one of vaccine antigens that induce non-pathogenic antibodies. The vaccine antigens inducing non-pathogenic antibodies are defined as “non-pathogenic vaccine antigens” or “safer vaccine antigens”, or “non-pathogenic antigens” of a pathogen hereafter in the present disclosure. The antibodies induced by non-pathogenic vaccine antigens or non-pathogenic antigens are defined as “non-pathogenic antibodies” hereafter in the present disclosure. The antigens of a pathogen inducing pathogenic antibodies are defined as “pathogenic antigens” hereafter in the present disclosure. Also provided are methods useful for identification of pathogenic and non-pathogenic antibodies inducible by a pathogen or the vaccines relating to the pathogen. Further provided are compositions comprising the safer vaccines, as well as polynucleotides, vectors, host cells, and methods useful in the production thereof. Further provided are methods and kits useful for treating or preventing infectious diseases in an individual by administering to the individual a safer vaccine capable of inducing non-pathogenic antibodies, optionally in combination with another vaccine.

In certain embodiments, the safer vaccines induce multivalent antibodies. In certain embodiments, the multivalent antibodies induced by the safer vaccines induce at least one of the non-pathogenic antibodies. In certain embodiments, the safer vaccines are mRNA vaccines. In certain embodiments, the safer vaccines are DNA vaccines. In certain embodiments, the safer vaccines are recombinant vaccines. In certain embodiments, the safer vaccines are virus vector vaccines. In certain embodiments, the safer vaccines are adenovirus vector vaccines. In certain embodiments, the safer vaccines are subunit vaccines. In certain embodiments, the safer vaccines are made from bacteria, or viruses. In certain embodiments the viruses are the respiratory viruses or enteroviruses. In certain embodiments, the respiratory viruses are selected from influenza viruses, respiratory enterovirus, adenovirus, coronavirus, rhinovirus, respiratory syncytial virus or B virus. In certain embodiments, the coronaviruses include the SARS-CoV-2 virus, SARS-CoV viruses, MERS-CoV viruses, and any variants or newly emerging strains of the coronaviruses. In certain embodiments, the influenza viruses are selected from type A, type B and type C influenza viruses. In certain embodiments, the influenza A viruses include at least one of H1N1, H3N2, H5N1, H7N9, H7N8 virus and any variants or newly emerging strains of the influenza viruses. In certain embodiments, the enteroviruses are selected from rotavirus, reovirus, Coxsackie virus, Echoviruses Enteroviruses, Polioviruses, norovirus, coronavirus, Norwalk virus, cytomegalovirus (CMV), herpes simplex virus, hepatitis virus, enteric cytopathic human orphan (ECHO) virus, porcine enterovirus (PEV), transmissible gastroenteritis virus (TGEV), foot and mouth disease (HFMD), human enterovirus 71, and porcine epidemic diarrhea virus (PEDV), and any variants or newly emerging strains of the enteroviruses.

In certain aspect, provided herein are safer vaccine antigens comprising at least one of the non-pathogenic antigens of a pathogen which induce non-pathogenic antibodies. In certain embodiments that may be combined with any of the preceding embodiments, the non-pathogenic vaccine antigens are selected from the surface or the outside proteins, or the surface or the outside glycoproteins, the envelope proteins, the envelope glycoproteins, the membrane proteins, the nucleocapsid proteins, the glycans of a pathogen. In certain embodiments, the non-pathogenic vaccine antigens are selected from any applicable antigens or saccharides of a pathogen, particular the pathogen antigens inducing non-pathogenic antibodies.

In certain embodiments that may be combined with any of the preceding embodiments, the non-pathogenic vaccine antigens are selected from viruses. In certain embodiments, the non-pathogenic vaccine antigens are selected from the coronaviruses including the SARS-CoV-2 virus, the SARS-CoV viruses, the MERS-CoV viruses, and any variants or newly emerging strains of the coronaviruses. In certain embodiments, the non-pathogenic vaccine antigens are selected from the influenza viruses including type A, type B and type C influenza viruses. In certain embodiments, the non-pathogenic vaccine antigens are selected from the influenza A viruses include at least one of H1N1, H3N2, H5N1, H7N9, H7N8 virus and any variants or newly emerging strains of the influenza viruses.

In certain embodiments that may be combined with any of the preceding embodiments, the non-pathogenic vaccine antigens are selected from the surface or the outside proteins, or the surface or the outside glycoproteins, the envelope proteins, the envelope glycoproteins, the membrane proteins, the nucleocapsid proteins, the glycans of a virus. In certain embodiments that may be combined with any of the preceding embodiments, the non-pathogenic vaccine antigens are selected from the spike protein, the envelope proteins, the spike glycoproteins, the glycans, the membrane proteins, the nucleocapsid proteins of the SARS-CoV-2 virus, the SARS-CoV-2 virus, the SARS-CoV viruses, the MERS-CoV viruses, and any variants or newly emerging strains of the coronaviruses. In certain embodiments, the non-pathogenic vaccine antigens are selected from the hemagglutinin (HA) proteins, the neuraminidase (NA) proteins, the other non-HA proteins, the envelope proteins, the envelope glycoproteins, the glycans, the capsid proteins and the nucleocapsid proteins of the influenza viruses.

In further aspects, the present disclosure provides an isolated polynucleotide comprising a nucleic acid sequence encoding a non-pathogenic vaccine antigen according to any of the above embodiments. In still further aspects, the present disclosure provides a vector comprising a nucleic acid sequence encoding a non-pathogenic vaccine antigen according to any of the above embodiments. In yet still further aspects, the present disclosure provides an isolated host cell comprising an isolated polynucleotide comprising a nucleic acid sequence encoding a non-pathogenic vaccine antigen according to any of the above embodiments or a vector comprising a nucleic acid sequence encoding a non-pathogenic vaccine antigen according to any of the above embodiments. In yet still further aspects, the present disclosure provides an isolated host cell comprising an isolated polynucleotide comprising a nucleic acid sequence encoding a non-pathogenic vaccine antigen according to any of the above embodiments or a vector comprising a nucleic acid sequence encoding a non-pathogenic vaccine antigen according to any of the above embodiments. In yet still further aspects, the present disclosure provides methods of producing a non-pathogenic vaccine antigen, comprising culturing a host cell according to any of the above embodiments, that produces the vaccine antigen according to any of the preceding embodiments, and recovering the non-pathogenic vaccine antigen from the cell culture. In yet still further aspects, the present disclosure provides a non-pathogenic vaccine antigen produced by the methods of producing a non-pathogenic vaccine antigen according to any of the above embodiments.

In other aspects, the present disclosure provides a composition comprising at least one of isolated polynucleotide comprising at least one of nucleic acid sequences encoding at least one of the non-pathogenic vaccine antigens according to any of the above embodiments, and a pharmaceutically acceptable carrier. In another aspect, the present disclosure provides a composition comprising at least one of vectors comprising at least one of nucleic acid sequences encoding at least one of the non-pathogenic vaccine antigens according to any of the above embodiments, and a pharmaceutically acceptable carrier. In other aspects, the present disclosure provides a composition comprising at least one of the non-pathogenic vaccine antigens according to any of the above embodiments, and a pharmaceutically acceptable carrier. In yet further aspects, the non-pathogenic vaccine antigens induce non-pathogenic antibodies.

In certain embodiments, the isolated polynucleotide comprising a nucleic acid sequence encoding a non-pathogenic vaccine antigen or a vector comprising a nucleic acid sequence encoding a non-pathogenic vaccine antigen is selected from viruses according to any of the above embodiments. In certain embodiments, an isolated polynucleotide comprising a nucleic acid sequence encoding a non-pathogenic vaccine antigen or a vector comprising a nucleic acid sequence encoding a non-pathogenic vaccine antigen is selected from the spike protein, the envelope proteins, the spike glycoproteins, the glycans, the membrane proteins, the nucleocapsid proteins of the SARS-CoV-2 virus, the SARS-CoV-2 virus, the SARS-CoV viruses, the MERS-CoV viruses, and any variants or newly emerging strains of the coronaviruses. In certain embodiments, an isolated polynucleotide comprising a nucleic acid sequence encoding a non-pathogenic vaccine antigen or a vector comprising a nucleic acid sequence encoding a non-pathogenic vaccine antigen is selected from the hemagglutinin (HA) proteins, the neuraminidase (NA) proteins, the other non-HA proteins, the envelope proteins, the envelope glycoproteins, the glycans, the capsid proteins and the nucleocapsid proteins of the influenza viruses. In yet further embodiments, the non-pathogenic vaccine antigens of the coronaviruses or the influenza viruses induce non-pathogenic antibodies.

In other aspects, the present disclosure provides a method for preventing or treating infectious diseases, infection-relating disease, and adverse reactions of vaccines or pathogenic antibodies in an individual, comprising administering to the individual an effective amount of a composition comprising a safer vaccine comprising at least one of isolated polynucleotide comprising at least one of nucleic acid sequences encoding at least one of the non-pathogenic vaccine antigens according to any of the above embodiments; or at least one of vectors comprising at least one of nucleic acid sequences encoding at least one of the non-pathogenic vaccine antigens according to any of the above embodiments, and a pharmaceutically acceptable carrier. In other aspects, the present disclosure provides a method for preventing or treating infectious diseases, infection-relating disease, and adverse reactions of vaccines or pathogenic antibodies in an individual, comprising administering to the individual an effective amount of a composition comprising a safer vaccine comprising at least one of the non-pathogenic vaccine antigens according to any of the above embodiments, and a pharmaceutically acceptable carrier. In yet still further aspects, the at least one of the non-pathogenic vaccine antigens encoded by the nucleic acid sequences induces non-pathogenic antibodies. In certain embodiments, the individual is a human. In certain embodiments, the individual is a non-human animal or organism.

In certain embodiments that may be combined with any of the preceding embodiments, the safer vaccine is a vaccine of a coronavirus including SARS-CoV-2 virus, SARS-CoV virus, MERS-CoV virus or a vaccine of an influenza virus. In certain embodiments that may be combined with any of the preceding embodiments, the safer vaccine of a coronavirus, or the safer vaccine of an influenza virus is a mRNA vaccine, a DNA vaccine, a recombinant vaccine, a viral vector vaccine, an adenovirus vector vaccine, a subunit vaccine, or any suitable or applicable types of vaccines.

In certain embodiments that may be combined with any of the preceding embodiments, the infectious diseases and the infection-relating diseases are caused by bacteria, or viruses, or other pathogenic organisms. In certain embodiments, the infectious diseases and the infection-relating diseases are caused by viruses as described in any of the preceding embodiments. In certain embodiments that may be combined with any of the preceding embodiments, the infectious diseases and the infection-relating diseases are caused by the enteroviruses. In certain embodiments that may be combined with any of the preceding embodiments, the infectious diseases and the infection-relating diseases are caused by respiratory viruses. In certain embodiments, the infectious diseases and the infection-relating diseases are caused by the coronaviruses including SARS-CoV-2 viruses, SARS-CoV viruses, MERS-CoV viruses, and any variants or newly emerging strains of the coronaviruses. In certain embodiments, the infectious diseases and the infection-relating diseases are caused by influenza viruses including type A, type B and type C influenza viruses. In certain embodiments, the infectious diseases are caused by the influenza A viruses include HINT, H3N2, H5NT, H7N9, H7N8 virus and any variants or newly emerging strains of the influenza viruses.

In certain embodiments that may be combined with any of the preceding embodiments, the adverse reactions of vaccines or pathogenic antibodies are caused the vaccines or the pathogenic antibodies inducible by bacteria, or by viruses, or by other pathogenic organisms. In certain embodiments, the adverse reactions of vaccines or pathogenic antibodies are caused by the vaccines or the pathogenic antibodies inducible by viruses as described in any of the preceding embodiments. In certain embodiments that may be combined with any of the preceding embodiments, the adverse reactions of vaccines or pathogenic antibodies are caused by the vaccines or the pathogenic antibodies inducible by respiratory viruses, or by the enteroviruses, as described in any of the preceding embodiments. In certain embodiments, the adverse reactions of vaccines or pathogenic antibodies are caused by the vaccines or the pathogenic antibodies inducible by the coronaviruses including SARS-CoV-2 viruses, SARS-CoV viruses, MERS-CoV viruses, and any variants or newly emerging strains of the coronaviruses. In certain embodiments, the adverse reactions of vaccines or pathogenic antibodies are caused by the vaccines or the pathogenic antibodies inducible by influenza viruses including type A, type B and type C influenza viruses. In certain embodiments, the adverse reactions of vaccines or pathogenic antibodies are caused by the vaccines or the pathogenic antibodies inducible by the influenza A viruses include HINT, H3N2, H5NT, H7N9, H7N8 virus and any variants or newly emerging strains of the influenza viruses.

In certain embodiments that may be combined with any of the preceding embodiments, the individual is a human, or a non-human animal. In certain embodiments that may be combined with any of the preceding embodiments, the safer vaccine is administered intramuscularly, subcutaneously, orally, by implantation, by inhalation, intranasally, or any suitable or applicable administrating route.

In other aspects, the present disclosure provides a method for making safer vaccines that induce at least one kind of non-pathogenic antibodies, comprising preparing a composition consisted of at least one of isolated polynucleotides comprising at least one of nucleic acid sequences encoding at least one of non-pathogenic vaccine antigens according to any of the above embodiments, or at least one of vectors comprising at least one of nucleic acid sequences encoding at least one of non-pathogenic vaccine antigens according to any of the above embodiments, and a pharmaceutically acceptable carrier. In further aspects, at least one of the non-pathogenic vaccine antigens encoded by the nucleic acid sequences induces non-pathogenic antibodies. In other aspects, the present disclosure provides a method for making a composition comprising at least one of the non-pathogenic vaccine antigens according to any of the above embodiments, and a pharmaceutically acceptable carrier. In yet still further aspects, the at least one of the non-pathogenic vaccine antigens according to any of the above embodiments induces non-pathogenic antibodies.

In certain embodiments that may be combined with any of the preceding embodiments, the safer vaccine is a vaccine of a coronavirus including a SARS-CoV-2 virus, a SARS-CoV virus, a MERS-CoV virus, and any variants or newly emerging strains of the coronavirus, or a vaccine of an influenza virus. In certain embodiments that may be combined with any of the preceding embodiments, the safer vaccine of the coronavirus, or the safer vaccine of an influenza virus is a mRNA vaccine, a DNA vaccine, a recombinant vaccine, a viral vector vaccine, an adenovirus vector vaccine, or a subunit vaccine, or any suitable or applicable types of applicable vaccines.

In other aspects, the present disclosure provides a kit comprising a pharmaceutical composition comprising a safer vaccine according to any of the above embodiments. In certain aspects, the kit further comprises instructions for administering an effective amount of the pharmaceutical composition to an individual for preventing an infectious disease, an infection-relating disease, or an adverse reaction of a vaccine or a pathogenic antibody. In some embodiments, the individual is at risk of an infection, an infection-relating disease, or an adverse reaction of a vaccine or a pathogenic antibody. In certain embodiments that may be combined with any of the preceding embodiments, the individual is a human or a non-human animal. In certain embodiments that may be combined with any of the preceding embodiments, the safer vaccine is administered intramuscularly, subcutaneously, orally, by implantation, by inhalation, intranasally, or any suitable or applicable administrating route.

In certain embodiments that may be combined with any of the preceding embodiments, the safer vaccine is a vaccine of a coronavirus including a SARS-CoV-2 virus, a SARS-CoV virus, a MERS-CoV virus, and any variants or newly emerging strains of the coronavirus, or a vaccine of an influenza virus. In certain embodiments that may be combined with any of the preceding embodiments, the safer vaccine of coronavirus, or the safer vaccine of an influenza virus is a mRNA vaccine, a DNA vaccine, a recombinant vaccine, a viral vector vaccine, an adenovirus vector vaccine, or a subunit vaccine, or any suitable or applicable types of applicable vaccines.

In other aspects, the present disclosure provides a kit comprising a pharmaceutical composition comprising at least one of non-pathogenic antibodies of a pathogen according to any of the above embodiments. In certain aspects, the kit further comprises instructions for administering an effective amount of the pharmaceutical composition to an individual for treating and preventing an infectious disease, an infection-relating disease, or an adverse reaction of a vaccine or a pathogenic antibody. In some embodiments, the individual is at risk of an infection, an infection-relating disease, or an adverse reaction of a vaccine or a pathogenic antibody, caused by the pathogen. In certain embodiments that may be combined with any of the preceding embodiments, the individual is a human or a non-human animal. In certain embodiments that may be combined with any of the preceding embodiments, the composition is administered intramuscularly, intravenously, intra-articularlly, intracerobrospinally, by infusion, intraperitoneally, subcutaneously, intrasynovialy, intrathecally, orally, by inhalation, intranasally, topically, and by any suitable or applicable administrating route.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows a timed-pregnant mouse model and the procedure of injection of anti-coronavirus antibodies into the model (A); the representative images of mouse pups delivered to the dames (B); and the sick and death rates of newborn mouse pups caused by the pathogenic anti-coronavirus antibodies, and the therapeutic effect of the mixed antibodies (C).

FIG. 2 shows the representative images of the histological changes of lungs (A-B), kidneys, brains and hearts (C-D) from the newborn mouse pups delivered to the dames injected with antibodies specific for the spike protein of either SARS-CoV-2 or SARS-CoV virus, the human anti-COVID-19 (SARS-CoV-2) spike monoclonal antibodies of B38 and REGN10987, and the control antibodies of human IgG and another human anti-COVID-19 (SARS-CoV-2) spike monoclonal antibody (MAb) of Cr3022-b6; or the dames treated in combination with other antibodies at the same time of the antibody injection of the anti-coronavirus antibodies. *2 MAbs: human monoclonal anti-COVID-19 (SARS-CoV-2) spike antibodies of CC12.3 and Cr3022-b6.

FIG. 3 shows the detection of the in vivo binding of anti-coronavirus spike antibodies at the inflammatory areas of the multiple organs of the mouse pups delivered to the dames with antibody injection at E15 and E18.

FIG. 4 shows the cytokine levels of MCP-1 and IL-4 of the mouse sera from the mouse pups delivered to the dames with the anti-coronavirus antibody injection alone or the dames treated in combination with other antibodies at the same time of the antibody injection of an anti-coronavirus antibody.

FIG. 5 shows the binding of anti-coronavirus spike or nucleocapsid antibodies and anti-influenza viral antibodies to the healthy and damaged human lung epithelium A549 cells without or with missed sialic acid.

FIG. 6 shows the binding of the human anti-COVID-19 (SARS-CoV-2) spike monoclonal antibody of Regn10987 to various human fetal tissues.

FIG. 7 shows the binding of the human anti-COVID-19 (SARS-CoV-2) spike monoclonal antibody of Regn10987 to various diseased human tissues of respiratory, cardiovascular, urinary system (A), and digestive system (B).

FIG. 8 shows the binding of the human anti-COVID-19 (SARS-CoV-2) spike monoclonal antibody of Regn10987 to various healthy human tissues.

### DETAILED DESCRIPTION

The present disclosure provides, safer vaccines comprising at least one of the vaccine antigens that induce non-pathogenic antibodies to a host. Multiple such safer vaccines are demonstrated herein to treat one or more infectious diseases in a variety of in vitro analysis and in vivo models.

In particularly, these safer vaccines were found to have the increased safeties as compared to existing vaccines, e.g., reduction of adverse reactions of the COVID-19 vaccines. In addition, a number of the non-pathogenic vaccine antigens were demonstrated the potential to reduce the serious adverse reactions of the COVID-19 vaccines representing a range of different types of the safer vaccines.

Provided herein are safer vaccines comprising at least one of the vaccine antigens that induce non-pathogenic antibodies. Also provided are methods useful for identification of pathogenic and non-pathogenic antibodies inducible by a pathogen or the vaccines relating to the pathogen. Further provided are compositions comprising the safer vaccines, as well as polynucleotides, vectors, host cells, and methods useful in the production thereof. Further provided are methods and kits useful for treating or preventing infectious diseases, infection-relating diseases and adverse reactions of vaccines or pathogenic antibodies in an individual, by administering to the individual a safer vaccine comprising at least one of the non-pathogenic vaccine antigens that induce non-pathogenic antibodies, optionally in combination with another vaccine.

### I. General Techniques

The techniques described or referenced herein are well understood and employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); Harlow and Lane, eds. (1988) Antibodies, A Laboratory Manual, and Animal Cell Culture (R. I. Freshney, ed. (1987)); Methods in Molecular Biology, Humana Press; Monoclonal Antibodies: A Practical Approach (P. Shepherd and C. Dean, eds., Oxford University Press, 2000); and Cancer: Principles and Practice of Oncology (V. T. DeVita et al., eds., J.B. Lippincott Company, 1993).

### II. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, the vaccine antigens inducing non-pathogenic antibodies are defined as “non-pathogenic vaccine antigens” or “safer vaccine antigens”, or “non-pathogenic antigens” of a pathogen. The antibodies induced by non-pathogenic vaccine antigens or non-pathogenic antigens are defined as “non-pathogenic antibodies”. The antigens of a pathogen inducing pathogenic antibodies are defined as “pathogenic antigens”. An antibody inducible by a pathogen or a vaccine of the pathogen that induces significant adverse reactions in a host is defined as a “pathogenic antibody”.

As used herein, the term “pathogens” refers to any organisms that can produce diseases. A pathogen may also be referred to as an infectious agent, or simply a germ. Typically, the term is used to describe an infectious microorganism or agent. Pathogens specific for infectious diseases include, but not limited to viruses, bacteria, parasites, fungi, viroids, prions, protozoa, and insects, etc. Types of pathogens include but not limited to any types of pathogens, live or dead or inactivated, fresh or dried, fixed or frozen, whole or part or fragment, sections, smears, homogenates, lysates, and extracts of pathogens. Examples of pathogens include but not limited to influenza viruses, coronaviruses such as SARS-CoV-2 virus, SARS-CoV virus, MERS-CoV virus, reoviruses, rotaviruses, cytomegaloviruses (CMV), Epstein-Barr viruses (EBV), adenoviruses, hepatitis viruses including HAV, HBV, HCV, human immunodeficiency virus (HIV), human T-cell leukemia viruses (HTLV), human papilloma viruses (HPV), polio viruses, parainfluenza viruses, measles viruses, mumps viruses, respiratory syncytial viruses (RSV), human herpes viruses (HHV), herpes simplex virus (HSV), Varicella-Zoster Virus, cholera viruses, pox virus, rabies virus, distemper virus, foot and mouth disease viruses, rhinoviruses, Newcastle disease viruses, pseudorabies virus, cholera, syphilis, anthrax, leprosy and bubonic plague, rickettsias, Neisseria gonorrhoeae, Bordetella pertussis, Escherichia coli, Salmonella enterica, Vibrio cholerae, Pseudomonas aeruginosa, Yersinia pestis, Francisella tularensis, Haemophilus influenzae, purple sulfur bacteria, Helicobacter pylori, Campylobacter jejuni, Bacillus anthracis/cereus/thuringiensis, Clostridium tetani, Clostridium botulinum, staphylococci, streptococci, pneumococci, Streptococcus pneumoniae, mycoplasmas, Bacteroides fragilis, Mycobacterium tuberculosis, Mycobacterium leprae, Corynebacterium diphtheriae, Treponema pallidum, Borrelia burgdorferi, Chlamydia trachomatis, Chlamydia psittaci, phycocyanin, phycoerythrin, mitochondria, chloroplasts.

As used herein, the term “saccharide” refers to a monosaccharide, an oligosaccharide or a polysaccharide. Monosaccharides include but not limited to fructose, glucose, mannose, fucose, xylose, galactose, lactose, N-acetylneuraminic acid, N-acetyl-galactosamine, N-acetylglucosamine, and sialic acids. An oligosaccharide is a saccharide polymer containing multiple sugar monomers linked by glycosidic linkages of component sugars.

Proteins are modified by the addition of saccharides, a process termed “protein glycosylation”. Glycoproteins or proteosaccharides refer to proteins linked with saccharides and may typically contain, for example, O- or N-glycosidic linkages of monosaccharides to compatible amino acid side chains in proteins or to lipid moieties. As used herein, the terms “glycan” and “glycosyl moiety” may be used interchangeably to refer to a saccharide alone or a sugar as the saccharide component of a glycoprotein. Two types of glycosylation are known in the art: N-linked glycosylation to the amide nitrogen of asparagine side chains and O-linked glycosylation to the hydroxy oxygen of serine and threonine side chains. Other saccharides include but not limited to O-GlcNAc, GAG Chain, glycosaminosaccharides, and glycosphinglipid. O- and N-linked saccharides are very common in eukaryotes but may also be found, although less commonly, in prokaryotes.

While many proteins are known to be glycosylated, glycoproteins are often found on the exterior surface of cells (i.e., extracellular) or secreted. Because of this, glycoproteins are highly accessible to external agents (e.g., exogenous compounds administered to a patient). For example, components that specifically recognize certain glycoproteins (e.g., antibodies or lectins) are able to bind, to an intact organism, to cells that express these glycoproteins on their cell surface. Components that specifically recognize certain glycoproteins are also able to bind secreted saccharides or glycoproteins, for example those that may be found freely in certain tissue samples (including in blood or serum).

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with cancer are mitigated or eliminated.

As used herein, the term “prevention” includes providing prophylaxis with respect to occurrence or recurrence of a disease in an individual. An individual may be predisposed to, susceptible to a type of cancer, or at risk of developing a type of cancer, but has not yet been diagnosed with the disease.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to effect a measurable improvement of a particular disorder (e.g., cancer). A therapeutically effective amount herein may vary according to factors such as the disease state, age, sex, and weight of the patient, and the ability of the monoclonal antibody to elicit a desired response in the individual. A therapeutically effective amount is also one in which any toxic or detrimental effects of the monoclonal antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in subjects prior to or at an earlier stage of disease, a prophylactically effective amount may be less than a therapeutically effective amount.

As used herein, administration “in conjunction” with another article or composition includes simultaneous administration and/or administration at different times. Administration in conjunction also encompasses administration as a co-formulation or administration as separate compositions, including at different dosing frequencies or intervals, and using the same route of administration or different routes of administration.

An “individual” for purposes of treatment or prevention refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human. In some embodiments, the individual is a non-human animal.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; low molecular weight (less than about 10 residues) polypeptide; proteins, such as serum albumin, gelatin, or immunoglobulins; amino acids such as glycine, glutamine, asparagine, arginine or lysine; carbohydrates including glucose, mannose, or dextrin; and/or nonionic surfactants such as TWEEN™, polyethylene glycol (PEG), and PLURONICS™.

“Pharmaceutically acceptable” buffers and salts include those derived from both acid and base addition salts of the above indicated acids and bases. Specific buffers and/or salts include histidine, succinate and acetate.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

An “isolated” polynucleotide encoding vaccine antigens herein is a nucleic acid molecule that is identified and separated from at least one contaminant nucleic acid molecule with which it is ordinarily associated in the environment in which it was produced. The isolated nucleic acid molecules encoding the polypeptides and vaccine antigens herein are in a form other than in the form or setting in which it is found in nature. Preferably, the isolated nucleic acid is free of association with all components associated with the production environment.

The term “vector,” as used herein, is intended to refer to a nucleic acid molecule capable of transporting another nucleic acid to which it has been linked. Types of vectors include plasmids (i.e., circular double stranded DNA into which additional DNA segments may be ligated) and viral vectors. Certain vectors are capable of autonomous replication in a host cell into which they are introduced (e.g., bacterial vectors having a bacterial origin of replication and episomal mammalian vectors). Other vectors can be integrated into the genome of a host cell and replicated along with the host genome. Moreover, certain vectors are capable of directing the expression of genes to which they are operatively linked. Such vectors are referred to herein as “recombinant expression vectors,” or simply, “expression vectors.” In the present specification, “plasmid” and “vector” may be used interchangeably as the plasmid is the most commonly used form of vector.

The term “antibody” herein is used in the broadest sense and specifically covers monoclonal antibodies, polyclonal antibodies, multi-specific antibodies (e.g., bispecific antibodies) formed from at least two intact antibodies, and antibody fragments (e.g., a Fab fragment, scFv, minibody, diabody, scFv multimer, or bispecific antibody fragment) so long as they exhibit the desired biological activity.

As used herein, the term “specifically binds to” or is “specific for” refers to measurable and reproducible interactions such as binding between a target and an antibody, that is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In certain embodiments, an antibody that specifically binds to a target has a dissociation constant (Kd) of ≤1 μM, ≤100 nM, ≤10 nM, ≤1 nM, or ≤0.1 nM. In another embodiment, specific binding can include, but does not require exclusive binding.

### III. Vaccines

### Vaccines

A “vaccine” is a biological preparation that provides active acquired immunity to a particular infectious disease. A vaccine typically contains an agent that resembles a disease-causing microorganism and is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future. Vaccines can be prophylactic (to prevent or ameliorate the effects of a future infection by a natural or “wild” pathogen), or therapeutic (to fight a disease that has already occurred, such as cancer). There are several types of vaccines, including: inactivated vaccines, live-attenuated vaccines, messenger RNA (mRNA) vaccines, subunit, recombinant, polysaccharide, and conjugate vaccines, toxoid vaccines, viral vector vaccines. While most vaccines are created using inactivated or attenuated compounds from micro-organisms, synthetic vaccines are composed mainly or wholly of synthetic peptides, carbohydrates, or antigens.

“Vaccination” refers to the administration of vaccines, usually as a shot. Vaccination is the most effective method of preventing infectious diseases. The effectiveness of vaccination has been widely studied and verified; for example, vaccines that have proven effective include the influenza vaccine, the HPV vaccine, the chicken pox vaccine, and recently the COVID-19 viral vaccine. The World Health Organization (WHO) reports that licensed vaccines are currently available for twenty-five different preventable infections.

“Messenger RNA vaccines” also called “mRNA vaccine” refers to a type of vaccine that uses a copy of a natural chemical called messenger RNA to produce an immune response. The vaccine transfects molecules of synthetic RNA into immunity cells. Once inside the immune cells, the vaccine's RNA functions as mRNA, causing the cells to build the foreign protein that would normally be produced by a pathogen or by a cancer cell. These protein molecules stimulate an adaptive immune response which teaches the body how to identify and destroy the corresponding pathogen or cancer cells. The delivery of mRNA is achieved by a co-formulation of the molecule into lipid nanoparticles which protect the RNA strands and helps their absorption into the cells. Among the COVID-19 vaccines are a number of RNA vaccines under development to combat the COVID-19 pandemic and some have received emergency use authorization in some countries.

“DNA vaccines” refers to a type of vaccine that insert and express viral or bacterial DNA in human or animal cells (enhanced by the use of electroporation), triggering immune system recognition. DNA vaccines work by injecting genetically engineered plasmid containing the DNA sequence encoding the antigen (s) specific for which an immune response is sought. Some cells of the immune system that recognize the proteins expressed will mount an attack specific for these proteins and cells expressing them. Because these cells live for a very long time, if the pathogen that normally expresses these proteins is encountered at a later time, they will be attacked instantly by the immune system. DNA vaccines have theoretical advantages over conventional vaccines, including the ability to induce a wider range of immune response types and easy to produce and store. The use of plasmids has been validated in preclinical studies as a protective vaccine strategy for cancer and infectious diseases. However, in human studies, this approach has failed to provide clinically relevant benefit. The overall efficacy of plasmid DNA immunization depends on increasing the plasmid's immunogenicity while also correcting for factors involved in the specific activation of immune effector cells.

“Viral vector vaccines” use a safe virus to insert pathogen genes in the body to produce specific antigens, such as surface proteins, to stimulate an immune response. Recombinant vector—by combining the physiology of one micro-organism and the DNA of another, immunity can be created specific for diseases that have complex infection processes.

A subunit vaccine uses a fragment of a micro-organism to create an immune response. One example is the subunit vaccine specific for hepatitis B, which is composed of only the surface proteins of the virus (now produced by recombination of the viral genes into yeast). Another example is edible algae vaccines, such as the virus-like particle (VLP) vaccine specific for human papillomavirus (HPV), which is composed of the viral major capsid protein. Another example is the hemagglutinin and neuraminidase subunits of the influenza virus.

Certain bacteria have a polysaccharide outer coat that is poorly immunogenic. By linking these outer coats to proteins (e.g., toxins), the immune system can be led to recognize the polysaccharide as if it were a protein antigen. This approach is used in the Haemophilus influenzae type B vaccine.^{[48]}

Subunit, recombinant, polysaccharide, and conjugate vaccines use specific pieces of the germ, like its protein, sugar, or capsid (a casing around the germ). Because these vaccines use only specific pieces of the germ, they give a very strong immune response that's targeted to the key parts of the germ.

Valence. Vaccines may be monovalent (also called univalent) or multivalent (also called polyvalent). A monovalent vaccine is designed to immunize specific for a single antigen or single microorganism. A multivalent or polyvalent vaccine is designed to immunize specific for two or more strains of the same microorganism, or specific for two or more microorganisms, or as used herein, two or more vaccine antigens of the same microorganism. In certain cases, a monovalent vaccine may be preferable for rapidly developing a strong immune response.

### IV. Pathogenic Antibodies

### Dualistic Roles of Anti-Pathogen Antibodies

Based on the traditional concept, the antibodies induced by an infectious pathogen or by a vaccine are protective to a host because they can neutralize the pathogen and prevent or treat the infectious disease. However, the roles of such antibodies can be dualistic. Some of the antibodies can cross react to certain cells, tissues, or organs of a host, triggers self-attack immune reactions such as antibody-dependent cytotoxicity (ADCC), or complement dependent cytotoxicity (CDC), or defects in signal transduction pathways, and cause injuries or disorders of the tissues and organs. For example, anti-viral antibodies can bind to host tissues and organs, irritate and cause injuries of the tissues and organs (e.g. autoimmune diseases) as described in PCT/US2009/039810 and PCT/US2014/25918. Further, compared to control mouse pups, administration of high dose of the anti-rotavirus antibodies to mouse pups before or after rotavirus infection caused deaths or severer infection of mouse pups as described in PCT/US2009/039810. In a mouse model of influenza infection, administration of high dose of the anti-2009H1N1 (swine) antibodies before viral infection caused deaths or severer infection of the mice compared to the control mice infected with virus alone, as described in PCT/US2014/25918.

A “mucous membrane” or “mucosa” is a membrane that lines body cavities and canals that lead to the outside, chiefly the respiratory, digestive, and urogenital tracts. It consists of one or more layers of epithelial cells overlying a layer of loose connective tissue. Mucous membranes also contain rich carbohydrates, predominantly glycoproteins or glycolipids. The oligosaccharide chains of membrane glycoproteins and glycolipids are formed by various combinations of six principal sugars D-galactose, D-mannose, L-fucose, N-acetylneuraminic acid (also called sialic acid), N-acetyl-D-glucosamine, and N-acetyl-D-galactosamine. The terminal sugar of the sugar chain, sialic acid, particularly N-acetylneuraminic acid has been found to be highly expressed on the surface of many types of mucous membrane and the surface of neural tissues. Sialic acid carries a negative charge, providing an external barrier to charged particles. The term mucous membrane comes from the fact that the major substance secreted from the membranes is mucus; the principal constituent of mucus is a mucopolysaccharide called mucin. Saccharides or glycans or sugar chains are predominant components of the mucus.

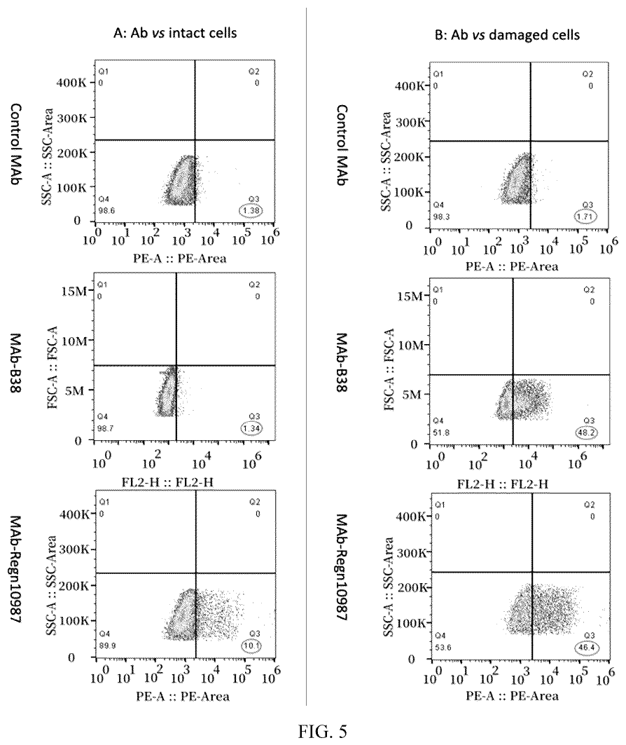

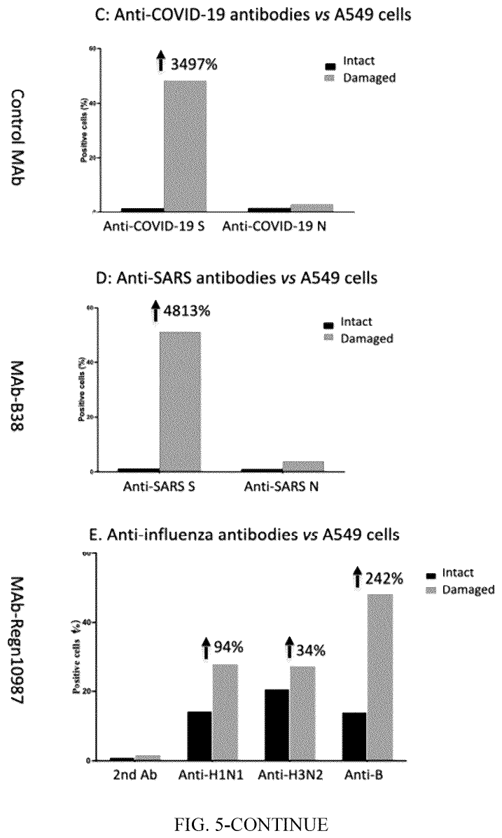

Sialic acids are predominant components of the mucous membrane at the out surface of cell membranes and mainly act as biological masks or receptors (Roland Schauer & Johannis P. Kamerling. Exploration of the sialic acid world. Elsevier, 2018, 12.1). Cells or tissues with sialic acid are recognized as “self”. After loss of sialic acids the cellular structures become “non-self” (R. Schauer & J. P. Kamerling. 2018) which can activate immune responses. During an infection of a pathogen (e.g. a virus) using sialic acid as an attachment molecule, the sialic acid on the infected cells (e.g. lung epithelium cells) could be removed or destroyed by the pathogens carrying sialidase (e.g. influenza viruses) or receptor destroy enzyme (RDE, e.g. coronavirus). The current invention discloses that certain antibodies specific for the spike protein of SARS-CoV-2 virus and SARS-CoV virus could significantly bind to the damaged lung epithelium cells and kidney embryonic cells with missed sialic acid on the cell surface, as shown in the Examples and FIG. 5.

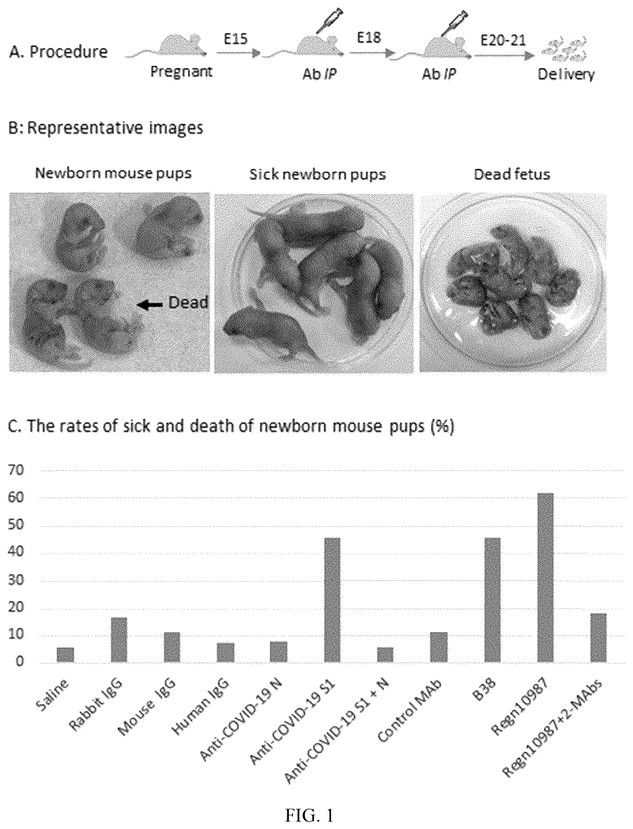

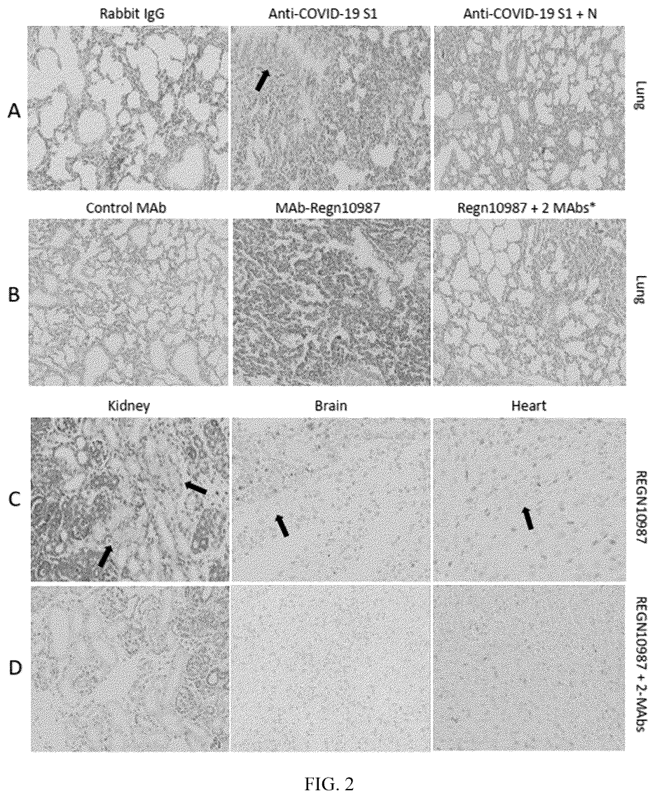

The antibody binding could mislead the immune response to attack self and induce the damage of multiple systems. For example, injection of high dose of the anti-rotavirus antibodies to pregnant mice induced deaths and bile duct epithelium proliferation (inflammation) of mouse pups born to the dames (PCT/US2009/039810); injection of human anti-influenza viral sera to pregnant mice induced fetal and neonatal deaths of mouse pups born to the dames (PCT/US2014/25918). Injection of the antibodies specific for the spike protein of SARS-CoV or SARS-CoV-2 virus (which causes the COVID-19 infection) to pregnant mice induced fetal and neonatal deaths of mouse pups born to the dames as described in Examples, FIGS. 1-3 and Table 1.

Therefore, based on the unexpected finding, one aspect of the present invention is to disclose a new concept of the pathogenic mechanisms of an infection. The in vitro and in vivo results support a new mechanisms of pathogenesis (MOP) of an highly pathogenic respiratory viral infection. The MOP include: 1) an highly pathogenic respiratory viruses such as the SARS-CoV-2 virus or the avian influenza virus causes the initial, primary injury such as local inflammation and cellular damage with missing sialic acids of its target organ (e.g. lung), typically within week one of the infection; 2) antibodies (e.g. anti-SARS-CoV-2 spike antibody) induced by the virus elevate and certain antibodies bind to the damaged and the inflammatory cells of the target organ and other organs with the similar injury (e.g. heart, brain and kidney), mislead the immune response to attack the self cells or tissues, and induces further damage (secondary injury); 3) the secondary damage persistently adds further injuries to the primary damage and cause serious conditions (e.g. ARDS) even death as the antibodies elevate and reach the peak levels from week one to weeks 2-4. 4) the overreacting immune responses (e.g. cytokine storm) mislead by the pathogenic antibodies can be persistent and accumulated after viral clearance whenever the antibody exist.

In certain embodiment, the primary injury is limited, short and decreased as the virus being cleared (such as a regular influenza infection). That means the virus itself is not enough to cause a serious condition such as ARDS or death. In other embodiment, the secondary injury caused by the pathogenic antibodies is longer and broader because antibodies persist much longer than viruses and can bind nonspecifically to other inflammatory tissues besides lung. In further embodiment, the new MOP are the reasons why most patients with serious respiratory viral infections such as COVID-19 or avian influenza infection died after one week especially at 2-4 weeks, matching the period of antibody peak levels.

In certain embodiment, the new MOP of an highly pathogenic viral infection are the reasons of the serious adverse reactions observed with the vaccines of respiratory viruses such as the COVID-19 vaccines and the influenza vaccines. In another embodiment, certain pathogenic antibodies inducible by other infectious pathogens or other vaccines also cause serious adverse reactions or autoimmune diseases through the similar pathogenic mechanism, even cancers if the inflammatory cellular proliferation stimulated by pathogenic antibodies loses control (e.g. cancers with HIV infected patients).

In certain embodiment, the majority (70% or more) of the anti-viral antibodies induced by either a virus or a vaccine is safe since the pathogenic antibodies of anti-COVID-19 S take less than 30%, according to a study with the monoclonal anti-S-RBD antibodies isolated from the patients infected with the COVID-19 virus, in which only 2/7 (28.6%) of the monoclonal anti-S-RBD antibodies caused significant adverse reactions.

### Pathogenic Antibodies and Pathogenic Antigens

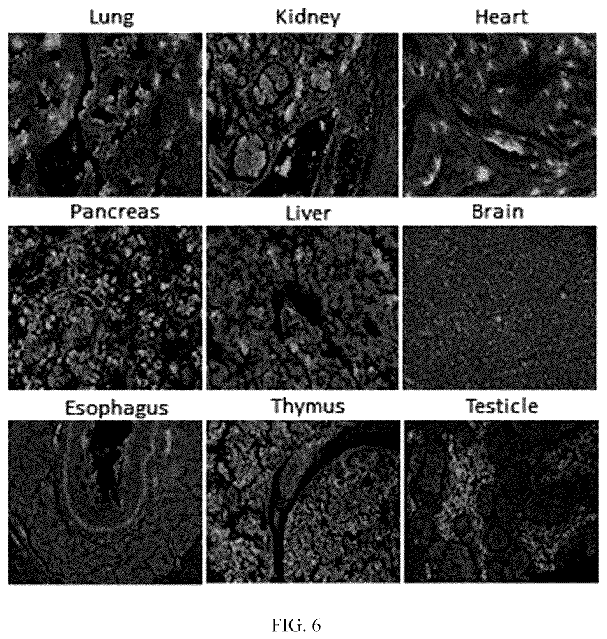

Certain aspect of the present invention is to disclose pathogenic antibodies or pathogenic antigens. In the present disclosure, the term “pathogenic antibodies” refers to any antibodies capable of causing pathogenic reactions and injuries or disorders of the cells, tissues and organs of a host. The pathogenic antibodies can be induced during an infection (e.g. an influenza infection or a coronavirus infection) or a vaccination (e.g. an influenza or a coronavirus vaccination), or passively introduced (e.g. a therapeutic antibody). The term “pathogenic antigens” refers to any antigens capable of inducing pathogenic antibodies, preferably a pathogenic antigen is from highly pathogenic infectious agents such as the COVID-19 virus or an avian influenza virus. In certain embodiments that may be combined with any of the preceding embodiments, the diseases or conditions caused by pathogenic antibodies or pathogenic antigens of the present disclosure, include but not limited to infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, infection-relating autoimmune diseases, allergy and infection-relating cancers, and any other disorders (known or unknown) inducible by pathogenic antibodies or pathogenic antigens. In addition, pathogenic antibodies can bind to the unmatured fetal cells or tissues (FIG. 6) and cause abortions, postpartum labors, still births of pregnant females, and neonatal deaths and neonatal sudden deaths, as shown in the Examples.

### Vulnerable Cells or Tissues to Pathogenic Antibodies

Another aspect of the present invention is to disclose cells or tissues vulnerable to pathogenic antibodies. “Vulnerable” herein means susceptible to an injury or a disease, or easy of being hurt. In certain embodiments that may be combined with any of the preceding embodiments, the vulnerable cells to pathogenic antibodies include but not limited to damaged or infected cells with missing sialic acid, inflammatory cells, actively proliferating cells and tumor cells, etc. In certain embodiments, the vulnerable cells to pathogenic antibodies are selected from the human adenocarcinoma alveolar basal epithelial cell line of A549 cells, human embryonic kidney (HEK) 293 cells, lung epithelial cell line of Beas-2B, human promyelocytic leukemia cell line of NB4. In certain embodiments, the vulnerable cells to pathogenic antibodies are selected from human blood cells including red blood cells, white blood cells and platelets. In certain embodiments, the vulnerable cells to pathogenic antibodies are selected from peripheral blood mononuclear cells (PBMCs). In certain embodiments, the vulnerable cells are selected from humans. In certain embodiments, the vulnerable cells are selected from non-human animals or non-human organisms. For example, binding of anti-SARS-CoV-2 viral antibodies and anti-SARS-CoV antibodies to healthy or damaged A549 cells are shown in the Examples and FIG. 5.

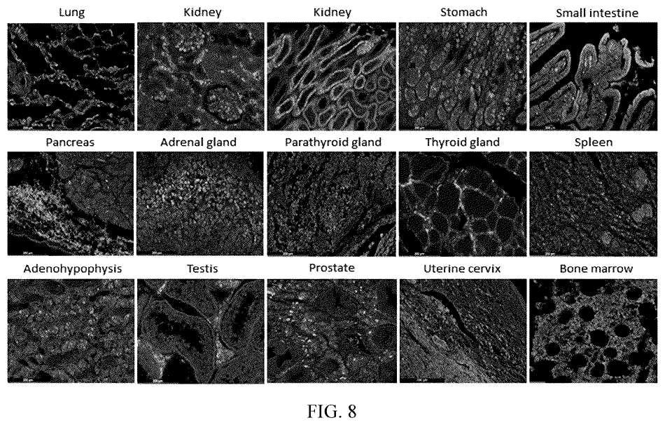

In certain embodiments that may be combined with any of the preceding embodiments, the vulnerable tissues to pathogenic antibodies include but not limited to fetal tissues. In certain embodiments, the vulnerable fetal tissues to pathogenic antibodies are selected from human fetal lung, heart, kidney, brain, pancreas, liver, intestine, thymus and testicle (FIG. 6). In certain embodiments that may be combined with any of the preceding embodiments, the vulnerable tissues to pathogenic antibodies include but not limited to human inflammatory tissues, infected tissues, or cancer tissues. In certain embodiments, the vulnerable tissues to pathogenic antibodies are selected from the diseases of human respiratory, cardiovascular, urinary system, neural and digestive system. In certain embodiments, the vulnerable tissues to pathogenic antibodies are selected from pneumonia, bronchitis, bronchiectasis, valvular disease, rheumatoid valvular disease, myocarditis, esophagitis, gastritis, colitis, appendicitis, pancreatitis, and hepatitis. In certain embodiment, the vulnerable tissues to pathogenic antibodies are selected from small cell lung carcinoma, kidney clear cell carcinoma, myxoma. In further embodiment, the vulnerable tissues to pathogenic antibodies are selected from the healthy human tissues of lung, kidney, pancreas, stomach, small intestine, spleen, bone marrow, adrenal gland, adenohypophysis, parathyroid gland, thyroid, testis, prostate, and uterine cervix of cancer adjacent cervical canals tissue (FIG. 8). In certain embodiments, the vulnerable tissues are selected from humans. In certain embodiments, the vulnerable tissues are selected from non-human animals or non-human organisms. For example, binding of an anti-SARS-CoV-2 spike antibody to human fetal tissues and various human diseased and healthy tissues are shown in the Examples and FIGS. 6-8.

In yet further embodiment that may be combined with any of the preceding embodiments, fetus or patients with pre-existing conditions are particularly vulnerable to a highly pathogenic infection or the vaccination of the pathogen, in which highly pathogenic antibodies are inducible during the infection or the vaccination. In certain embodiment that may be combined with any of the preceding embodiments, the pre-existing conditions are chronic inflammatory diseases, autoimmune diseases, or cancers. In yet more embodiments that may be combined with any of the preceding embodiments, the pathogenic antibodies induced by a pathogen during an infection or by a vaccine during a vaccination, bind to the vulnerable cells or tissues, rapidly activate immune responses to attack the antibody-bound cells or tissues, and cause serious adverse reactions. In certain embodiments, the vulnerable fetus or patients are humans. In certain embodiments, the vulnerable fetus or patients are non-human animals or non-human organisms. A pregnant mouse fetal model vulnerable to the pathogenic antibodies specific for COVID-19 spike protein are shown in the Examples and FIGS. 1-4.

### Methods for Identification of Pathogenic and Non-Pathogenic Antibodies

Certain aspects of the present disclosure relate to the methods for identification of pathogenic or non-pathogenic antibodies inducible by a pathogen or by a vaccine relating to the pathogen.

In Vitro Assay with Cultured Cells for Screening Potential Pathogenic Antibodies

One aspect of the present disclosure relates to an in vitro assay with cultured cells comprising:
a) treating a selected cultured cells with neuraminidase or sialidase for a period time sufficient for the sialidase's effective function;
b) binding a selected antibody specific for a pathogen or a vaccine to the cells with or without sialidase treatment;
c) washing off the free antibodies and detecting the presence of the antibodies on the surface of the cells.
d) a selected antibody significantly bind to the damaged cells with missing sialic acid has the potential to bind the diseased cells in vivo during the infection of the pathogen or the vaccination of the vaccines relating to the pathogen and causes pathogenic reactions, the antibody will be selected as potential pathogenic antibody and to be proceeded to the in vivo test.

In certain embodiment that may be combined with any of the preceding embodiments, the selected cells are vulnerable to an infectious pathogen. In certain embodiment that may be combined with any of the preceding embodiments, the selected cells are from the target organ or cells (e.g. lung epithelium cells) of an infectious pathogen (e.g. COVID-19 virus). In certain embodiments that may be combined with any of the preceding embodiments, the selected antibody is inducible by an infectious pathogen or a vaccine relating to the pathogen. In certain embodiment, the detecting assay for detection of the presence of the antibodies on the cells is a flow cytometry assay, an ELISA assay, and an immunofluorescence assay. In certain embodiment, the detecting assay of the present disclosure also contain any other reagents useful for the antibody detection, such as 96-well microtiter plates, a non-specific protein such as bovine serum albumin, a secondary antibody that binds to an selected antibody of the present disclosure without affecting its antigen-binding, and reagents for detection, such as a fluorescent or luminescent label, or an enzyme and substrate that produce a detectable signal (e.g., horseradish peroxidase and TMB).

One aspect of the present disclosure relates to a potential pathogenic antibody identified by the in vitro assay with cultured cells. In certain embodiments that may be combined with any of the preceding embodiments, the potential pathogenic antibody significantly binds to the damaged cells with missing sialic acid on the cellular surface. For example, binding of anti-coronavirus antibodies to healthy (intact) or damaged lung epithelium cells was tested with the human lung epithelium cell line A549 and the in vitro assay as described in Examples and FIG. 5. For another example, two human monoclonal antibodies specific for the COVID-19 spike protein, strongly bound to the damaged A549 cells with missed sialic acid. A control antibody also specific for the COVID-19 spike protein did not bind to the healthy A549 cells nor the damaged cells (FIG. 5A). For more examples, the antibodies specific for the spike glycoprotein of SARS-CoV virus (anti-SARS S) strongly bound to the damaged A549 cells with missed sialic acid while the antibody did not bind to the healthy A549 cells with sialic acid (FIG. 5D). For further example, the polyclonal antibody specific for SARS-CoV-2 nucleocapsid protein (anti-COVID-19 N) and the antibody specific for SARS-CoV nucleocapsid protein (anti-SARS N) did not significantly bind to neither healthy nor damaged A549 cells (FIG. 5C-D). Therefore, the antibodies specific for the spike proteins of the COVID-19 virus are “potential pathogenic antibodies” because they have the potential to cause pathogenic reactions in vivo. The antibodies specific for the nucleocapsid proteins of the COVID-19 virus and the SARS virus are “potential nor-pathogenic antibodies”. Some of the potential pathogenic and non-pathogenic antibodies were selected to proceed to an in vivo test for further confirmation.

### In Vitro Antibody Binding to Diseased or Healthy Tissues

Another aspect of the present disclosure relates to another in vitro assay with diseased or/and healthy tissues comprising binding a selected antibody specific for a pathogen or a vaccine to diseased or/and healthy tissues, and detecting the presence of the antibodies on the surface of the diseased or/and healthy tissues. A selected antibody significantly binds to a diseased or/and a healthy tissue in vitro has the potential to bind the similar diseased or healthy tissue in vivo during the infection or the vaccination of the pathogen and causes pathogenic reactions. The antibody will be a potential pathogenic antibody and selected to proceed to the in vivo test. In certain embodiment, the diseased or/and healthy tissues are selected from humans. In certain embodiment, the diseased or/and healthy tissues are selected from human blood cells including red blood cells, white blood cells and platelets. In certain embodiment, the diseased or/and healthy tissues are selected from non-human animals.

In certain embodiment, the diseased or/and healthy tissues is vulnerable to an infectious pathogen. In certain embodiment, the diseased or/and healthy tissues is selected from the target organ (e.g. lung) of an infectious pathogen (e.g. COVID-19 virus). In certain embodiments that may be combined with any of the preceding embodiments, the selected antibody is inducible by an infectious pathogen or a vaccine relating to the pathogen. In certain embodiment, the detecting assay for detection of the presence of the antibodies the diseased or/and healthy tissues is a tissue array, an immunohistochemistry assay, an immunofluorescence assay, a flow cytometry assay, and an ELISA assay. In certain embodiment, the detecting assay of the present disclosure also contain any other reagents useful for the antibody detection, such as 96-well microtiter plates, a non-specific protein such as bovine serum albumin, a secondary antibody that binds to an selected antibody of the present disclosure without affecting its antigen-binding, and reagents for detection, such as a fluorescent or luminescent label, or an enzyme and substrate that produce a detectable signal (e.g., horseradish peroxidase and TMB).

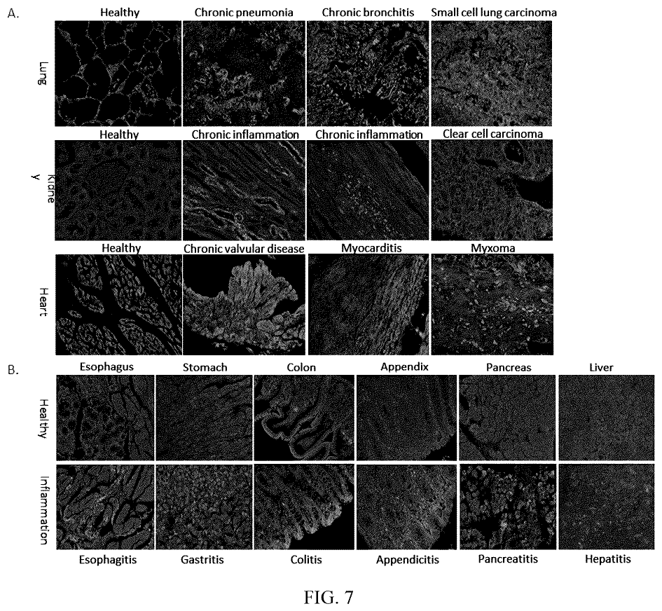

Certain aspect of the present disclosure relates to a potential pathogenic antibody identified by the in vitro antibody binding assay with the diseased or/and healthy tissues. In certain embodiments that may be combined with any of the preceding embodiments, the potential pathogenic antibody binds to diseased or/and healthy tissues. In certain embodiments that may be combined with any of the preceding embodiments, the potential pathogenic antibody binds to fetal tissues. For example, binding of an anti-COVID-19 S-RBD antibody to human fetal tissues and various human diseased tissues are described in Examples and shown in FIGS. 6-8. The antibody strongly bound to the damaged A549 cells with missed sialic acid (FIG. 5), also bound broadly to the human fetal tissues (FIG. 6) and human inflammatory tissues or cancer tissues of respiratory, cardiovascular, urinary and digestive system (FIG. 7). In certain embodiment, the antibody bound to the human fetal tissues selected from lung, heart, kidney, brain, pancreas, liver, intestine, thymus and testicle (FIG. 6). In certain embodiment, the antibody bound to the human inflammatory diseases selected from pneumonia, bronchitis, bronchiectasis, valvular disease, rheumatoid valvular disease, myocarditis, esophagitis, gastritis, colitis, appendicitis, pancreatitis, and hepatitis (FIG. 7). In certain embodiment, the antibody bound to the human cancer tissues selected from small cell lung carcinoma, kidney clear cell carcinoma, myxoma, (FIG. 7). In further embodiment, the antibody bound to the healthy human tissues of lung, kidney, pancreas, stomach, small intestine, spleen, bone marrow, adrenal gland, adenohypophysis, parathyroid gland, thyroid, testis, prostate, and uterine cervix of cancer adjacent cervical canals tissue (FIG. 8). Therefore, the antibody has the highly potential to cause seriously pathogenic reactions in vivo and was selected to proceed to an in vivo test. The data further indicated that the most of the inflammatory disease tissues or some of cancer tissues are vulnerable to a pathogenic antibody.

### Animal Models for Confirmation of Pathogenic and Non-Pathogenic Antibodies

One aspect of the present invention discloses the experimental models by administering anti-pathogen antibodies into non-human animals for confirmation the pathogenicity of pathogenic antibodies or non-pathogenicity of non-pathogenic antibodies. In certain embodiments, the non-human animals are selected from chicken embryos or pregnant mice or newborn mouse pups. Among the previous patent applications of PCT/US2014/25918 (Biological therapeutics for infectious or inflammatory diseases or conditions) published animal models of chicken embryos or pregnant mice or newborn mouse pups for pathogenic studies and evaluating the safety of vaccines and antibodies such as anti-influenza viral vaccines and antibodies. The timed-pregnant mouse model was used in the current disclosure for identification of the pathogenic antibodies specific for the coronaviruses including the SARS-CoV-2 virus.

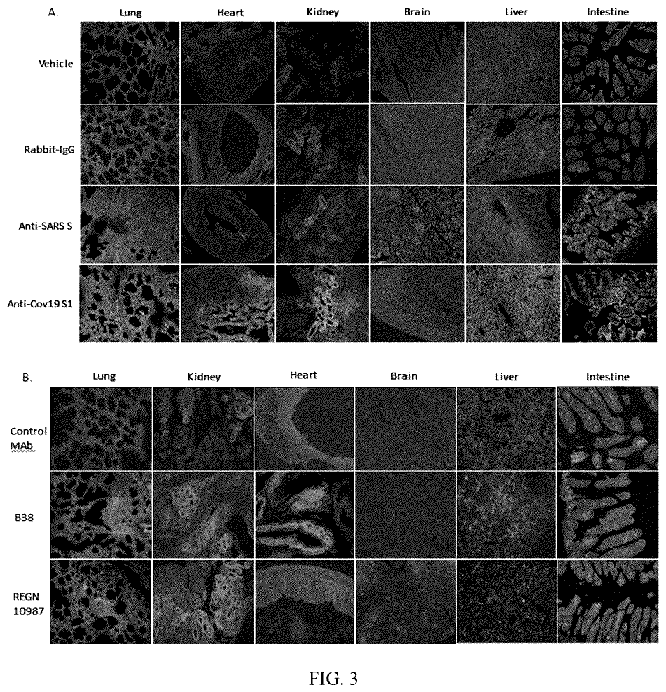

One aspect of the present invention is to disclose the experimental model which is developed by administering anti-coronavirus antibodies into pregnant mice and observing the healthy status of the newborn mouse pups as described in the Examples. As shown in FIG. 1, injection of the antibodies specific to COVID-19 S1 or COVID-19 S-RBD antigens into pregnant mice at pregnant day 15 (E15) and E18, induced immature or delayed delivery, and fatal and neonatal death of the newborn mouse pups delivered to the dames. The frequencies of sick and death of the newborn mouse pups are listed in Table 1 and FIG. 1C. The highly potential pathogenic antibody specific for the COVID-19 S-RBD as described in the preceding embodiments, induced the highest frequency of sick and death of the newborn mouse pups (FIG. 1C and Table 1) and tissue lesions of lungs, brains, hearts and kidneys (FIGS. 2-4). In contrast, the antibodies specific for the nucleocapsid proteins of the COVID-19 virus and the SARS virus did not induce significant sick and death neither tissue lesions (FIGS. 1-4, Table 1). Thus, the antibody specific for the COVID-19 S-RBD was confirmed through the in vivo test as a “highly pathogenic antibody”. The antibodies specific for the nucleocapsid proteins of the COVID-19 virus and the SARS virus was confirmed through the in vivo test as “non-pathogenic antibodies”.

In certain embodiments that may be combined with any of the preceding embodiments, the confirmed pathogenic antibody is specific for SARS-CoV-2 S1 antigens. In certain embodiments that may be combined with any of the preceding embodiments, the confirmed pathogenic antibody is specific for the SARS-CoV S antigens. In certain embodiments that may be combined with any of the preceding embodiments, the pathogenic antibody is specific for the SARS-CoV-2 S-RBD antigens. In certain embodiments that may be combined with any of the preceding embodiments, the pathogenic antibody is specific for the other parts of the SARS-CoV-2 spike proteins. In certain embodiments that may be combined with any of the preceding embodiments, the pathogenic antibody is specific for the spike proteins of the SARS-CoV or the MERS-CoV viruses, or the other coronaviruses. In certain embodiments that may be combined with any of the preceding embodiments, the pathogenic antibody is specific for the envelope proteins, the envelope glycoproteins, the membrane proteins, the glycans, and any applicable antigens or saccharides of the SARS-CoV-2 virus, or the SARS-CoV or the MERS-CoV viruses, or any other applicable coronaviruses.

In certain embodiments that may be combined with any of the preceding embodiments, the pathogenic antibody is specific for the hemagglutinin (HA) proteins, the envelope proteins, the envelope glycoproteins, the glycans, and the capsid proteins and any applicable antigens or saccharides of the influenza viruses. In certain embodiments that may be combined with any of the preceding embodiments, the pathogenic antibody is specific for the surface or the outside proteins, the surface or the outside glycoproteins, the envelope proteins, the envelope glycoproteins, the membrane proteins, the glycans, and any applicable antigens or saccharides of a pathogen. In certain embodiments that may be combined with any of the preceding embodiments, the pathogens are bacteria. In certain embodiments that may be combined with any of the preceding embodiments, the pathogens are viruses.

In certain embodiments that may be combined with any of the preceding embodiments, the non-pathogenic antibodies are specific for the SARS-CoV-2 S-RBD antigens. In certain embodiments that may be combined with any of the preceding embodiments, the confirmed non-pathogenic antibodies are specific for the nucleocapsid antigens of SARS-CoV-2 virus or the SARS-CoV virus. In certain embodiments that may be combined with any of the preceding embodiments, the non-pathogenic antibodies are specific for the nucleocapsid antigens of MERS-CoV virus and other coronaviruses. In other embodiments that may be combined with any of the preceding embodiments, the non-pathogenic antibodies are specific for the envelope protein, the envelope glycoprotein antigens, the membrane protein antigens, the glycans, and any applicable antigens or saccharides of the SARS-CoV-2 virus, or the SARS-CoV or the MERS-CoV viruses, or any other applicable coronaviruses. In more embodiments that may be combined with any of the preceding embodiments, the non-pathogenic antibodies are specific for the spike antigens of the SARS-CoV-2 virus, or the SARS-CoV or the MERS-CoV viruses, or any other applicable coronaviruses, which do not induce pathogenic antibodies.

In certain embodiments that may be combined with any of the preceding embodiments, the non-pathogenic antibodies are specific for the neuraminidase (NA) proteins of the influenza viruses. In certain embodiments that may be combined with any of the preceding embodiments, the non-pathogenic antibodies are specific for the envelope proteins, the envelope glycoproteins, the glycans, the capsid proteins, the other non-HA proteins, and any applicable antigens or saccharides of the influenza viruses. In more embodiments that may be combined with any of the preceding embodiments, the non-pathogenic antibodies are specific for hemagglutinin (HA) proteins of the influenza viruses which do not induce pathogenic antibodies.

In yet further embodiments that may be combined with any of the preceding embodiments, the non-pathogenic antibodies are specific for the nucleocapsid antigens of a pathogen. In certain embodiments that may be combined with any of the preceding embodiments, the non-pathogenic antibodies are specific for the surface or the outside proteins, or the surface or the outside glycoproteins of a pathogen. In certain embodiments that may be combined with any of the preceding embodiments, the non-pathogenic antibodies are specific for the envelope proteins of a pathogen. In certain embodiments, the non-pathogenic antibodies are specific for the envelope glycoprotein of a pathogen. In certain embodiments, the non-pathogenic antibodies are specific for the glycans of a pathogen. In certain embodiments, the non-pathogenic antibodies are specific for the membrane proteins of a pathogen. In certain embodiments, the non-pathogenic antibodies are specific for any applicable antigens or saccharides of a pathogen, which do not induce pathogenic antibodies. In certain embodiments that may be combined with any of the preceding embodiments, the pathogens are bacteria. In certain embodiments that may be combined with any of the preceding embodiments, the pathogens are viruses.

In yet further embodiments that may be combined with any of the preceding embodiments, the present invention is to disclose another experimental model which is developed by administering anti-pathogen antibodies into chicken embryos and observing the healthy status of the newborn chicks as described in the previous patent applications of PCT/US2014/25918 (Biological therapeutics for infectious or inflammatory diseases or conditions). In certain embodiments, the anti-pathogen antibodies cause adverse reactions of the embryos or newborn chicks. In certain embodiments, the anti-pathogen antibodies cause death of the embryos or newborn chicks. In certain embodiments, the anti-pathogen antibodies cause Guillain-Barre syndrome (GBS) or GBS-like condition of the newborn chicks. The anti-pathogen antibodies causing adverse reactions of the embryos or newborn chicks are identified as pathogenic antibodies. The anti-pathogen antibodies not causing significant adverse reactions of the embryos or newborn chicks are identified as non-pathogenic antibodies.

One aspect of the present invention is to disclose the uses of the methods for identification of pathogenic or non-pathogenic antibodies. In certain embodiments that may be combined with any of the preceding embodiments, the methods for identification of pathogenic or non-pathogenic antibodies of the present disclosure are used but not limited for the pathogenic study of infectious diseases, autoimmune diseases (e.g. GBS or GBS-like condition), infection-relating diseases (e.g. infection relating fetal and neonatal deaths), adverse reactions of vaccines (e.g. COVID-19 vaccines or influenza vaccines), or therapeutic antibodies. In another embodiment that may be combined with any of the preceding embodiments, the methods for identification of pathogenic or non-pathogenic antibodies of the present disclosure are used for rapid evaluating the safety of a vaccine or therapeutic antibodies, and screening safer vaccine antigens. In another embodiment that may be combined with any of the preceding embodiments, the methods for identification of pathogenic or non-pathogenic antibodies of the present disclosure are used for screening drugs for prevention and treatment of the disorders or conditions caused by pathogenic antibodies. Other embodiments besides the above may be articulated as well. Numerous other objects, features and advantages of the present disclosure will become readily apparent from the detailed description of the methods for identification of pathogenic or non-pathogenic antibodies of the present disclosure.

### V. Production of Safer Vaccines

Certain aspects of the present disclosure relate to the methods for manufacturing safer vaccines. In some embodiments, the safer vaccines induce monovalent antibody specific for a non-pathogenic antigen of a pathogen. In some embodiments, the safer vaccines induce multivalent antibodies comprising at least one kind of non-pathogenic antibodies. In some embodiments, the safer vaccines induce multivalent antibodies specific for two different epitopes of one antigen of a pathogen, in which at least one epitope of the antigen induces non-pathogenic antibodies. In some embodiments, the safer vaccines induce multivalent antibodies specific for at least two different antigens of a pathogen in which at least one antigen of the pathogen induces non-pathogenic antibodies.

### Polynucleotides, Vectors Encoding Vaccine Antigens, and Host Cells

Certain aspects of the present disclosure relate to the production of safer vaccines that produce at least one of non-pathogenic antibodies. In particularly, certain aspects relate to isolated polynucleotides containing a nucleic acid sequence encoding a safer vaccine antigen or a non-pathogenic vaccine antigen that produce a non-pathogenic antibody. Polynucleotides may refer to deoxyribonucleotides, ribonucleotides, modified nucleotides or bases, and/or their analogs. These polynucleotides may be produced in vivo in a host cell or through in vitro transcription. Polynucleotides encoding a safer vaccine antigen may refer to polynucleotides bearing the sequence encoding the vaccine antigen as it was identified in a cell producing the vaccine antigen, or polynucleotides containing synonymous mutations in the sequence that distinguish them from their naturally occurring counterparts but, due to the inherent degeneracy of the genetic code, encode a similar protein. Polynucleotides may be isolated by any means known in the art, including PCR followed by precipitation-based purification of the PCR reaction, or a slice of agarose gel containing the PCR product, or by purification of a vector containing the polynucleotide from a host cell (e.g., plasmid preparation from E. coli).

Certain aspects of the present disclosure relate to vectors containing a nucleic acid sequence encoding a safer vaccine antigen that produce a non-pathogenic antibody. For recombinant production of vaccine antigens or fragments thereof, nucleic acids encoding the desired vaccine antigens or vaccine antigen fragments are isolated and inserted into a replicable vector for further cloning (amplification of the DNA) or for expression. DNA encoding the vaccine antigen is readily isolated (e.g., with oligonucleotide probes that specifically bind to genes encoding the vaccine antigen) and sequenced using conventional procedures. Many cloning and/or expression vectors are commercially available.

Vector components generally include, but are not limited to, one or more of the following, a signal sequence, an origin of replication, one or more marker genes, a multiple cloning site containing recognition sequences for numerous restriction endonucleases, an enhancer element, a promoter, and a transcription termination sequence. Both expression and cloning vectors contain a nucleic acid sequence that enables the vector to replicate in one or more selected host-cells. Such sequences are well known for a variety of bacteria, yeast, and viruses. Expression and cloning vectors may also contain a selection gene, known as a selectable marker, whose expression confers resistance to antibiotics or other toxins, complements auxotrophic deficiencies, or supplies critical nutrients not available from complex media.

Expression and cloning vectors usually contain a promoter that is recognized by the host organism and is operably linked to the nucleic acid encoding the vaccine antigens (e.g., nucleocapsid protein of SARS-CoV-2 virus) or fragments thereof. Promoters suitable for use with prokaryotic hosts include the phoA promoter, lactamase and lactose promoter systems, alkaline phosphatase promoter, a tryptophan promoter system, and hybrid promoters such as the tac promoter, although other known bacterial promoters are also suitable. Promoters for use in bacterial systems also will contain a Shine-Dalgamo (S.D.) sequence operably linked to the DNA encoding the vaccine antigens and fragments thereof. Promoter sequences are known for eukaryotes, including the yeast promoters for 3-phosphoglycerate kinase or other glycolytic enzymes and mammalian promoters obtained from the genomes of viruses such as polyoma virus, cytomegalovirus, and most preferably Simian Virus 40 (SV40). Various heterologous mammalian promoters, e.g., the actin promoter, and heat-shock promoters, are also known. Expression vectors used in eukaryotic host cells will also contain sequences necessary for the termination of transcription and for stabilizing the mRNA.

Certain aspects of the present disclosure relate to isolated host cells with vectors containing a nucleic acid sequence encoding a safer vaccine antigen that produce a non-pathogenic antibody. Suitable host-cells for cloning or expressing the DNA encoding vaccine antigen s (e.g., nucleocapsid protein of SARS-CoV-2 virus) or fragments thereof in the vectors described herein prokaryotes such as Gram-negative or Gram-positive organisms, for example, Enterobacteriaceae such as E. coli. In addition to prokaryotes, eukaryotic microbes such as filamentous fungi or yeast are also suitable cloning or expression hosts, such as Saccharomyces cerevisiae. For a review discussing the use of yeasts and filamentous fungi for the production of therapeutic proteins, see, e.g., Gerngross, Nat. Biotech. 22: 1409-1414 (2004). Suitable host-cells for the expression of glycosylated vaccine antigens or the fragments of vaccine antigens are derived from multicellular organisms. Examples of invertebrate cells include plant and insect-cells such as Spodoptera frugiperda (caterpillar), Aedes aegypti (mosquito), Drosophila melanogaster (fruitfly), or Bombyx mori (moth) cells. Examples of useful mammalian host-cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Nat'l Acad. Sci. USA 77:4216 (1980)); African green monkey kidney cells (VERO-76, ATCC CRL-1587); human cervical carcinoma cells (HELA, ATCC CCL 2); and a human hepatoma line (Hep G2). For a review of certain mammalian host cell lines suitable for vaccine antigen production, see, e.g., Yazaki and Wu, Methods in Molecular Biology, Vol. 248 (B. K. C. Lo, ed., Humana Press, Totowa, N J, 2003), pp. 255-268. These examples are illustrative rather than limiting.

Certain aspects of the present disclosure relate to convert the recombinant DNA as described above into mRNA to make a mRNA vaccine. The mRNA comprises a nucleic acid sequence encoding a safer vaccine antigen that produce a non-pathogenic antibody. A variety of modification techniques have been used to produce more stable mRNA and enhance protein translation. Examples of the techniques include but not limited to replacing natural RNA with synthetic non natural RNA, synthesizing the “cap” like structure and “capping enzymes”, adding 5′cap, 3′ poly (a) “tail” and UTR (untranslated region) sequences, and modifying nucleotide to reduce innate immune activation. Among the modification techniques, there are 16 modifications found on eukaryotic mRNA, 13 of which have been included in RNA modification database (mamdb). These modifications can be divided into methylation, pseudouracil and hypoxanthine. The main modifications of mammalian mRNA are N1- and N6-methyladenosine (m1a, m6A), 3- and 5-methylcytosine (m3c, m5C), 5-hydroxymethylcytosine (hm5c), and pseudouridine (Ψ) And 2′-O-methylation (nm). In the development of mRNA vaccine, the main modification methods are N6-methyladenosine and pseudouridine (Ψ), and 2′-O-methylation (nm). N6 methyladenosine (m6A) can regulate the stability of mRNA. However, the immune response of human body to mRNA vaccine is mainly related to uridine (partly composed of uracil). Using pseudouracil instead of uracil can reduce the recognition of mRNA by immune system. The 2′-O-methylation modification of RNA 5′ cap can make it escape the host's antiviral response.

General mRNA isolation and/or purification techniques include but not limited to RNase III treatment and fast protein liquid chromatography (FPLC) purification. The synthesized mRNA is wrap or encapsulated in a delivery carrier (e,g, a liposome) for delivery to its cell destination. The delivery carrier of mRNA vaccine mainly includes liposome, non liposome, virus and nanoparticles.

Vaccine antigen production and purification Certain aspects of the present disclosure relate to methods of producing a vaccine antigen or a pathogenic antigen of a pathogen thereof by culturing host cells with vectors containing a nucleic acid sequence encoding a vaccine antigen or fragments thereof and recovering the vaccine antigen from the cell culture. Host cells are transformed with the above-described expression or cloning vectors for vaccine antigen or vaccine antigen fragment production and cultured in conventional nutrient media modified as appropriate for inducing promoters, selecting transformants, or amplifying the genes encoding the desired sequences. Either a safer vaccine antigen or fragments or a pathogenic antigen can be made by the methods. The safer vaccine antigens or fragments can be used to make safer subunit vaccines of a pathogen. The pathogenic antigens of a pathogen can be used as therapeutics to neutralize the pathogenic antibodies induced by the pathogen. In certain embodiments, pathogenic antigens of a pathogen are fragments, synthetic peptides, glycans, glycoproteins, proteins of pathogens of any of the preceding embodiments,

The host-cells used to produce the safer vaccine antigens or the pathogenic antigens of a pathogen (e.g., SARS-CoV-2 virus) or the antigen fragments, described herein may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host-cells. Any of these media may be supplemented as necessary with hormones and/or other growth factors (such as insulin, transferrin, or epidermal growth factor), salts (such as sodium chloride, calcium, magnesium, and phosphate), buffers, nucleotides, antibiotics, trace elements, and glucose or an equivalent energy source. Any other necessary supplements may also be included at appropriate concentrations that would be known to those skilled in the art. The culture conditions, such as temperature, pH, and the like, are those previously used with the host-cell selected for expression and will be apparent to the ordinarily skilled artisan.

When using recombinant techniques, the vaccine antigens (e.g., nucleocapsid protein of SARS-CoV-2 virus) or the antigen fragments of the pathogen can be produced intracellularly, in the periplasmic space, or secreted directly into the medium. Vaccine antigens prepared from such cells can be purified using, for example, hydroxylapatite chromatography, gel electrophoresis, dialysis, and affinity chromatography, such as that using protein A or protein G attached to a matrix (e.g., agarose). In general, various methodologies for purifying preparing vaccine antigens for use in research, testing, and clinical applications are well-established in the art, consistent with the above-described methodologies and/or as deemed appropriate by one skilled in the art for a particular vaccine antigen of interest.

In certain embodiments that may be combined with any of the preceding embodiments, the safer vaccine antigens or the non-pathogenic vaccine antigens are selected from the surface or the outside proteins, or the surface or the outside glycoproteins, the envelope proteins, the envelope glycoprotein, the glycans, membrane proteins, the nucleocapsid proteins, and any applicable antigens or saccharides of a pathogen, particularly the safer vaccine antigens induce non-pathogenic antibodies. In other embodiments that may be combined with any of the preceding embodiments, the pathogenic antigens of a pathogen are selected from the surface or the outside proteins, or the surface or the outside glycoproteins, the envelope proteins, the envelope glycoprotein, the glycans, membrane proteins, and any applicable antigens or saccharides of the pathogen, particularly the pathogenic antigens can neutralize the pathogenic antibodies induced by the pathogen.

In certain embodiments that may be combined with any of the preceding embodiments, the safer vaccine antigens or the pathogenic antigens are selected from bacteria. In certain embodiments that may be combined with any of the preceding embodiments, the safer vaccine antigens or the pathogenic antigens are selected from viruses. In certain embodiments, the safer vaccine antigens or the pathogenic antigens are selected from the coronaviruses including the SARS-CoV-2 virus, SARS-CoV viruses, MERS-CoV viruses, and any variants or newly emerging strains of the coronaviruses. In certain embodiments, the pathogenic antigens are selected from the spike proteins of the SARS-CoV-2 virus, SARS-CoV viruses, MERS-CoV viruses, and any variants or newly emerging strains of the coronaviruses. In certain embodiments, the pathogenic antigens are selected from the receptor binding domain (RBD) of the spike proteins (S-RBD) of the SARS-CoV-2 virus, SARS-CoV viruses, MERS-CoV viruses, and any variants or newly emerging strains of the coronaviruses. In certain embodiments, the safer vaccine antigens or the pathogenic antigens are selected from the influenza viruses including type A, type B and type C influenza viruses. In certain embodiments, the safer vaccine antigens are selected from the influenza A viruses include at least one of H1N1, H3N2, H5N1, H7N9, H7N8 virus and any variants or newly emerging strains of the influenza viruses.

In certain embodiments that may be combined with any of the preceding embodiments, the safer vaccine antigens or the pathogenic antigens are selected from the surface or the outside proteins, or the surface or the outside glycoproteins, the envelope proteins, the envelope glycoprotein, the glycans, membrane proteins, the nucleocapsid proteins, and any applicable antigens or saccharides of a virus. In certain embodiments, the safer vaccine antigens or the pathogenic antigens are selected from the envelope proteins, the spike proteins, the spike glycoproteins, the glycans, the membrane proteins, the nucleocapsid proteins, and any applicable antigens or saccharides of the SARS-CoV-2 virus, the SARS-CoV virus, the MERA-CoV virus, and other coronaviruses. In certain embodiments, the pathogenic antigens are selected from the spike proteins of the SARS-CoV-2 virus, SARS-CoV viruses, MERS-CoV viruses, and any variants or newly emerging strains of the coronaviruses. In certain embodiments, the pathogenic antigens are selected from the receptor binding domain (RBD) of the spike proteins (S-RBD) of the SARS-CoV-2 virus, SARS-CoV viruses, MERS-CoV viruses, and any variants or newly emerging strains of the coronaviruses. In certain embodiments, the safer vaccine antigens or the pathogenic antigens are selected from the hemagglutinin (HA) proteins, the neuraminidase (NA) proteins, the other non-HA proteins, the envelope proteins, the envelope glycoproteins, the glycans, the capsid proteins and the nucleocapsid proteins of the influenza viruses.

### Compositions Containing Safer Vaccines or Pathogenic Antigens

Certain aspects of the present disclosure relate to composition containing safer vaccine antigens or pathogenic antigens of a pathogen. In some embodiments, compositions containing safer vaccine antigens of a pathogen may include pharmaceutically acceptable adjuvants, carriers, excipients, or stabilizers that are nontoxic to the cell or mammal being exposed thereto at the dosages and concentrations employed as part of a pharmaceutical composition. In general, various vaccine adjuvants for use are well-established in the art. Examples of vaccine adjuvants include aluminum, monophosphoryl lipid A (MPL), oil-in-water emulsion composed of squalene, and cytosine phosphoguanine (CpG). Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrin; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

Certain aspects of the present disclosure relate to composition containing pathogenic antigens of a pathogen. In certain embodiments, the pathogenic antigens of a pathogen are fragments, synthetic peptides, glycans, glycoproteins, proteins of pathogens of any of the preceding embodiments. In some embodiments, compositions containing pathogenic antigens of a pathogen may include pharmaceutically acceptable carriers, excipients, or stabilizers that are nontoxic to the cell or mammal being exposed thereto at the dosages and concentrations employed as part of a pharmaceutical composition.

In certain embodiments that may be combined with any of the preceding embodiments, a composition containing safer vaccine antigens or pathogenic antigens of a bacteria. In certain embodiments that may be combined with any of the preceding embodiments, a composition containing safer vaccine antigens or pathogenic antigens of a virus. In certain embodiments that may be combined with any of the preceding embodiments, a composition containing safer vaccine antigens or pathogenic antigens of a coronavirus including the SARS-CoV-2 virus, the SARS-CoV virus, the MERA-CoV virus, and other coronaviruses. In certain embodiments, the pathogenic antigens are selected from the spike proteins of the SARS-CoV-2 virus, SARS-CoV viruses, MERS-CoV viruses, and any variants or newly emerging strains of the coronaviruses. In certain embodiments, the pathogenic antigens are selected from the receptor binding domain (RBD) of the spike proteins (S-RBD) of the SARS-CoV-2 virus, SARS-CoV viruses, MERS-CoV viruses, and any variants or newly emerging strains of the coronaviruses. In certain embodiments that may be combined with any of the preceding embodiments, a composition containing safer vaccine antigens or pathogenic antigens of an influenza virus including type A, type B and type C influenza viruses, in which the influenza A viruses include at least one of HIN1, H3N2, H5NT, H7N9, H7N8 virus and any variants or newly emerging strains of the influenza viruses.

### VI. Methods of Treatment

### Infectious Diseases

As used herein, the term “infectious diseases” refers to the invasion of a host organism's bodily tissues by disease-causing organisms, their multiplication, and the reaction of host tissues to these organisms and the toxins they produce. A short-term infection is an acute infection. A long-term infection is a chronic infection. Pathogens specific to infectious diseases suitable for use in this process include but not limited to viruses, bacteria, parasites, fungi, viroids, prions, protozoa, and insects, and etc., without limitation. Examples of infections include but not limited to the disorders caused by influenza viruses, coronaviruses, reoviruses, rotaviruses, cytomegaloviruses (CMV), Epstein-Barr viruses (EBV), adenoviruses, hepatitis viruses including HAV, HBV, HCV, human immunodeficiency virus (HIV), human T-cell leukemia viruses (HTLV), human papilloma viruses (HPV), polio viruses, parainfluenza viruses, measles viruses, mumps viruses, respiratory syncytial viruses (RSV), human herpes viruses (HHV), herpes simplex virus (HSV), Varicella-Zoster Virus, cholera viruses, pox virus, rabies virus, distemper virus, foot and mouth disease viruses, rhinoviruses, Newcastle disease viruses, pseudorabies virus, cholera, syphilis, anthrax, leprosy and bubonic plague, rickettsias, Neisseria gonorrhoeae, Bordetella pertussis, Escherichia coli, Salmonella enterica, Vibrio cholerae, Pseudomonas aeruginosa, Yersinia pestis, Francisella tularensis, Haemophilus influenzae, purple sulfur bacteria, Helicobacter pylori, Campylobacter jejuni, Bacillus anthracis/cereus/thuringiensis, Clostridium tetani, Clostridium botulinum, staphylococci, streptococci, pneumococci, Streptococcus pneumoniae, mycoplasmas, Bacteroides fragilis, Mycobacterium tuberculosis, Mycobacterium leprae, Corynebacterium diphtheriae, Treponema pallidum, Borrelia burgdorferi, Chlamydia trachomatis, Chlamydia psittaci, phycocyanin, phycoerythrin, mitochondria, chloroplasts, etc without limitation.

### Infection-Relating Diseases

As used herein, the term “infection-relating diseases” refers to the disorders or conditions occurred during or after an infection. According to the present invention, infection-relating diseases or conditions include but not limited to the complications or sequela of infections, autoimmune diseases, allergies, inflammation and tumors occurred during or after an infection. The disorders or conditions usually arise after a period time (e.g. within 2-6 weeks) of an infection. Examples of infection-relating autoimmune diseases, allergies, inflammation and tumors include but not limited to cytokine storm, cytokine release syndrome, Guillain-Barre syndrome, autism, Kawasaki's disease, biliary atresia, primary biliary cirrhosis, systemic lupus erythematous, leukemia, acute leukemia, rheumatoid arthritis, adult onset diabetes mellitus (Type II diabetes), Sjogren's syndrome, juvenile onset diabetes mellitus, Hodgkin's and non-Hodgkin's lymphoma, malignant melanoma, cryoglobulinemia, inflammatory bowel disease, polymyositis, dermatomyositis, multiple endocrine failure, Schmidt's syndrome, autoimmune uveitis, Addison's disease, adrenalitis, Graves' disease, thyroiditis, Hashimoto's thyroiditis, autoimmune thyroid disease, subacute cutaneous lupus erythematosus, hypoparathyroidism, autoimmune thrombocytopenia, autoimmune hemolytic anemia, dermatitis herpetiformis, autoimmune cystitis, male or female autoimmune infertility, ankylosing spondylitis, ulcerative colitis, Crohn's disease, mixed connective tissue disease, polyarteritis nodosa, systemic necrotizing vasculitis, juvenile onset rheumatoid arthritis, rheumatic fever, asthma, recurrent abortion, Behcet's disease, endocarditis, myocarditis, endomyocardial fibrosis, endophthalmitis, Alzheimer's disease, post vaccination syndromes, and any other disorder or conditions in which the specific recognition of the host by pathogen-inducible or vaccine-inducible antibodies is suspected or shown to be important in any aspect of the pathogenesis of the clinical illness. The infection-relating diseases further include but not limited to abortion, postpartum labor, still birth of the pregnant females, and neonatal death and neonatal sudden death, caused by an infection or by a vaccine.

In certain embodiments that may be combined with any of the preceding embodiments, the infectious diseases or the infection-relating diseases are caused by bacteria, or viruses, or other pathogenic organisms. In certain embodiments, the infectious diseases or the infection-relating diseases are caused by viruses as described in any of the preceding embodiments. In certain embodiments that may be combined with any of the preceding embodiments, the infectious diseases or the infection-relating diseases are caused by the enteroviruses. In certain embodiments that may be combined with any of the preceding embodiments, the infectious diseases or the infection-relating diseases are caused by respiratory viruses. In certain embodiments, the infectious diseases or the infection-relating diseases are caused by the coronaviruses including SARS-CoV-2 viruses, SARS-CoV viruses, MERS-CoV viruses, and any variants or newly emerging strains of the coronaviruses. In certain embodiments, the infectious diseases or the infection-relating diseases are caused by influenza viruses including type A, type B and type C influenza viruses. In certain embodiments, the infectious diseases are caused by the influenza A viruses include H1N1, H3N2, H5N1, H7N9, H7N8 virus and any variants or newly emerging strains of the influenza viruses.

### Complications or Sequela of Infections

The complication of an infection refers to the disorders or conditions occurred during the infection. The sequela of an infection refers to the disorders or conditions occurred after the infection. The complications or the sequela of the COVID-19 infection or an highly pathogenic influenza infection or other infections include but not limited to acute respiratory failure, pneumonia, acute respiratory distress syndrome (ARDS), acute kidney injury, acute cardiac injury, acute liver injury, acute injury of neural system, Bell's palsy, secondary infection, septic shock, blood clots, disseminated intravascular coagulation, multisystem inflammatory syndrome in children, chronic fatigue, fibrotic lung, new-onset diabetes, stroke, heart attack, new-onset epilepsy, psychological illness, easy clotting/thrombosis, high fever, swelling and redness, extreme fatigue, nausea, Acute Disseminated Encephalomyelitis (ADEM), Guillian Barre Syndrome (GBS), meningitis, encephalitis, rhabdomyolysis, cytokine storms, cytokine release syndrome, bacteremia, sepsis, bronchitis, sinutis, enlarged tonsils, tonsillitis, swollen lymph nodes (bull neck), myocarditis, infectious mononucleosis, heart attacks, strokes, high fever, swelling and redness, extreme fatigue, nausea, cytokine storms, autoimmune diseases, deaths, etc.

### COVID-19 Long Hauler

COVID-19 symptoms can last weeks or months for some people. These patients, given the name “long haulers”, have in theory recovered from the worst impacts of COVID-19 and have tested negative. However, they still have symptoms. The most common long hauler symptoms include but not limited to coughing, ongoing, sometimes debilitating, fatigue, body aches, joint pain, shortness of breath, loss of taste and smelt difficulty sleeping, headaches, brain fog, etc. Brain fog refers to unusually forgetful, confused or unable to concentrate even enough to watch TV (Marshall, M. The lasting misery of coronavirus long-haulers. Nature 585, 339-341, 2020).

### Adverse Reactions of Vaccines or Pathogenic Antibodies

As used herein, the term “adverse reactions” of vaccines or pathogenic antibodies of the present disclosure refers to the severe disorders or conditions caused by pathogenic antibodies induced by a vaccine during a vaccination. The vaccines include but not limited to the vaccines of bacteria, viruses and all the pathogens according to any of the above embodiments. The vaccines of viruses include but not limited to influenza viruses, coronaviruses including SARS, SARS-CoV-2 and MERS, and all the viruses according to any of the above embodiments. The serious disorders or conditions usually arise after a period time (e.g. from day 3 to week 4) of a vaccination matching the period of the peak levels of the produced antibodies. Examples of serious adverse reactions of vaccines of the present disclosure include but not limited to deaths, ARDS, coagulation abnormality, thrombocytopenia, stroke, blood clots, disseminated intravascular coagulation, Bell's palsy, acute infant death syndrome, cytokine storm, cytokine release syndrome, Guillain-Barre syndrome, Kawasaki's disease, acute leukemia, allergies, serious allergic reactions, asthma, epilepsy, immune system disorders, behavior disorders, nervous system disorders or injury, permanent brain damage, learning difficulties, seizure, severe seizures, lowered consciousness, autism, long-term coma, headaches, upper or low respiratory tract infection, joint pain, abdominal pain, cough, nausea, diarrhea, high fever, blood in the urine or stool, pneumonia, inflammation of the stomach or intestines, non-stop crying, fainting, deafness, temporary low platelet count, hives, pain in the joints, intussusception, vomiting, severe nervous system reaction, life-threatening severe illness with organ failure, still birth, neonatal deaths, and any other disorder or conditions in which an infection of the host is suspected or shown to be important in any aspect of the pathogenesis of the clinical illness, blood clots, disseminated intravascular coagulation, heart attacks, etc.

Certain aspects of the present disclosure provide methods for preventing or treating infectious diseases, infection-relating diseases, complications or sequela of infections, COVID-19 long hauler, adverse reactions of vaccines and pathogenic antibodies in an individual according to any of the above embodiments, comprising administering to the individual an effective amount of a composition comprising a safer vaccine or a pathogenic antigen of the pathogen causing the above conditions. In certain embodiments that may be combined with any of the preceding embodiments, the individual is a human, or a non-human animal, or another organism. In certain embodiments that may be combined with any of the preceding embodiments, the safer vaccines are administered intramuscularly, subcutaneously, orally, by implantation, by inhalation, intranasally, or any suitable or applicable administrating route. In certain embodiments that may be combined with any of the preceding embodiments, the safer vaccines induce less adverse reactions, particular the serious adverse reactions. In certain embodiments that may be combined with any of the preceding embodiments, the pathogenic antigens are administered intravenously, intramuscularly, subcutaneously, topically, orally, transdermally, intraperitoneally, intraorbitally, by implantation, by inhalation, intrathecally, intraventricularly, or intranasally, or any suitable or applicable administrating route. In certain embodiments that may be combined with any of the preceding embodiments, the pathogenic antigens neutralize the pathogenic antibodies induced by the pathogen.

In certain embodiments that may be combined with any of the preceding embodiments, the safer vaccines are mRNA vaccines, DNA vaccines, recombinant vaccines, viral vector vaccines, adenovirus vector vaccines, subunit vaccines, or any suitable or applicable types of applicable vaccines. In certain embodiments that may be combined with any of the preceding embodiments, the safer vaccines are vaccines of bacteria. In certain embodiments that may be combined with any of the preceding embodiments, the safer vaccines are vaccines of viruses. In certain embodiments that may be combined with any of the preceding embodiments, the safer vaccines are vaccines of coronavirus including the SARS-CoV-2 virus, SARS-CoV viruses, MERS-CoV viruses, and any variants or newly emerging strains of the coronaviruses. In certain embodiments that may be combined with any of the preceding embodiments, the safer vaccines are vaccines of influenza viruses including type A, type B and type C influenza viruses. In certain embodiments, the safer vaccines are vaccines of the influenza A viruses include at least one of H1N1, H3N2, H5N1, H7N9, H7N8 virus and any variants or newly emerging strains of the influenza viruses.

In certain embodiments that may be combined with any of the preceding embodiments, the pathogenic antigens of a pathogen are recombinant antigens, fragment antigens, subunit antigens, synthetic peptides, glycans, glycoproteins, proteins or any suitable or applicable types of applicable antigens of the pathogen which are capable of neutralizing pathogenic antibodies inducible by the pathogen but do not inducing antibodies. In certain embodiments that may be combined with any of the preceding embodiments, the pathogenic antigens are selected from bacteria. In certain embodiments that may be combined with any of the preceding embodiments, the pathogenic antigens are selected from viruses. In certain embodiments that may be combined with any of the preceding embodiments, the pathogenic antigens are selected from coronavirus including the SARS-CoV-2 virus, SARS-CoV viruses, MERS-CoV viruses, and any variants or newly emerging strains of the coronaviruses. In certain embodiments that may be combined with any of the preceding embodiments, the pathogenic antigens are selected from influenza viruses including type A, type B and type C influenza viruses. In certain embodiments, the pathogenic antigens are selected from the influenza A viruses include at least one of H1N1, H3N2, H5N1, H7N9, H7N8 virus and any variants or newly emerging strains of the influenza viruses. In certain embodiments, the pathogenic antigens of a pathogen can neutralize the pathogenic antibodies induced by the pathogen.

Certain aspects of the present disclosure provide methods for preventing or treating infectious diseases, infection-relating diseases, complications or sequela of infections, COVID-19 long hauler, adverse reactions of vaccines and pathogenic antibodies in an individual according to any of the above embodiments, comprising administering to the individual an effective amount of a composition comprising non-pathogenic antibodies. In certain embodiments that may be combined with any of the preceding embodiments, the non-pathogenic antibodies are administered intramuscularly, intravenously, intra-articularlly, intracerobrospinally, by infusion, intraperitoneally, subcutaneously, intrasynovialy, intrathecally, orally, by inhalation, intranasally, and topically, or by any suitable or applicable administrating route. In certain embodiments that may be combined with any of the preceding embodiments, the non-pathogenic antibodies do not induce significant adverse reactions, particular not serious adverse reactions. In certain embodiments that may be combined with any of the preceding embodiments, the individual is infected with an infectious pathogen of any one of the preceding embodiments. In certain embodiments that may be combined with any of the preceding embodiments, the individual is vaccinated with a vaccine relating to any one of the infectious pathogens of any one of the preceding embodiments. In certain embodiments that may be combined with any of the preceding embodiments, the individual is a human. In certain embodiments that may be combined with any of the preceding embodiments, the individual is a non-human animal, or another organism.

In certain embodiments that may be combined with any of the preceding embodiments, the non-pathogenic antibodies are specific for the non-pathogenic antigens or the safer vaccine antigens of a pathogen of any one of the preceding embodiments. In certain embodiments that may be combined with any of the preceding embodiments, the non-pathogenic antibodies are specific for the nucleocapsid proteins of the SARS-CoV-2 virus, or SARS-CoV viruses. In certain embodiments that may be combined with any of the preceding embodiments, the non-pathogenic antibodies are specific for the S-RBD proteins of the SARS-CoV-2 virus. In certain embodiments that may be combined with any one of the preceding embodiments, the non-pathogenic antibodies are specific for the neuraminidase (NA) proteins, the non-HA proteins, the envelope proteins, the envelope glycoproteins, the capsid proteins and the nucleocapsid proteins of the influenza viruses.

### VI. Kits

Certain aspects of the present disclosure relate to kits containing a pharmaceutical composition containing a safer vaccine that produce at least one of non-pathogenic antibodies. In some embodiments, the kits may further include instructions for administering an effective amount of the pharmaceutical composition to an individual for preventing infectious diseases, infection-relating diseases, adverse reactions of vaccines or pathogenic antibodies. These instructions may refer to instructions customarily included in commercial packages of medicaments that contain information about the indications, usage, dosage, administration, contraindications, other medicaments to be combined with the packaged product, and/or warnings concerning the use of such medicaments, etc.

Certain aspects of the present disclosure relate to kits containing a pharmaceutical composition containing a pathogenic antigen of a pathogen that neutralize the pathogenic antibodies induced by the pathogen. In some embodiments, the kits may further include instructions for administering an effective amount of the pharmaceutical composition to an individual for preventing infectious diseases, infection-relating diseases, adverse reactions of vaccines or pathogenic antibodies. These instructions may refer to instructions customarily included in commercial packages of medicaments that contain information about the indications, usage, dosage, administration, contraindications, other medicaments to be combined with the packaged product, and/or warnings concerning the use of such medicaments, etc.

Certain aspects of the present disclosure relate to kits containing a pharmaceutical composition containing non-pathogenic antibodies. In some embodiments, the kits may further include instructions for administering an effective amount of the pharmaceutical composition to an individual for treating or preventing infectious diseases, infection-relating diseases, adverse reactions of vaccines or pathogenic antibodies. These instructions may refer to instructions customarily included in commercial packages of medicaments that contain information about the indications, usage, dosage, administration, contraindications, other medicaments to be combined with the packaged product, and/or warnings concerning the use of such medicaments, etc.

Suitable containers for a kit of the present disclosure include, for example, bottles, vials (e.g., dual chamber vials), syringes (such as single or dual chamber syringes) and test tubes. The article of manufacture may further comprise a label or a package insert, which is on or associated with the container, may indicate directions for reconstitution and/or use of the formulation. The label or package insert may further indicate that the formulation is useful or intended for injection or other modes of administration for preventing infectious diseases in an individual. The article of manufacture may further include other materials desirable from a commercial, therapeutic, and user standpoint, including other buffers, diluents, filters, needles, syringes, and package inserts with instructions for use.

Certain aspects of the present disclosure relate to kits containing a composition containing pathogenic antigens of pathogens of any one of the preceding embodiments, and instructions or other reagents for using the composition for detecting the presence of a pathogenic antibody of a pathogen or a vaccine in a biological sample of an individual. Any body-fluid or secretion may be used as a biological sample of the present disclosure. Examples of biological samples may include without limitation blood, serum, urine, feces, milk, semen, saliva, chest fluid, abdominal fluid, cerebrospinal fluid, sputum, and any other body fluid or secretion.

In certain embodiments that may be combined with any one of the preceding embodiments, the individual is infected with an infectious pathogen of any one of the preceding embodiments. In certain embodiments that may be combined with any of the preceding embodiments, the individual is infected with a coronavirus including the SARS-CoV-2 virus, SARS-CoV viruses, MERS-CoV viruses, and any variants or newly emerging strains of the coronaviruses. In certain embodiments that may be combined with any of the preceding embodiments, the individual is infected with an influenza virus including type A, type B and type C influenza viruses. In certain embodiments, the safer vaccines are vaccines of the influenza A viruses include at least one of HIN1, H3N2, H5N1, H7N9, H7N8 virus and any variants or newly emerging strains of the influenza viruses. In certain embodiments that may be combined with any of the preceding embodiments, the individual is vaccinated with a vaccine relating to any one of the infectious pathogens of any one of the preceding embodiments. In certain embodiments that may be combined with any of the preceding embodiments, the individual is vaccinated with a coronavirus including the SARS-CoV-2 virus, SARS-CoV viruses, MERS-CoV viruses, and any variants or newly emerging strains of the coronaviruses. In certain embodiments that may be combined with any of the preceding embodiments, the individual is vaccinated with an influenza virus including type A, type B and type C influenza viruses. In certain embodiments, the safer vaccines are vaccines of the influenza A viruses include at least one of H1N1, H3N2, H5N1, H7N9, H7N8 virus and any variants or newly emerging strains of the influenza viruses. In certain embodiments that may be combined with any of the preceding embodiments, the individual is a human. In certain embodiments that may be combined with any of the preceding embodiments, the individual is a non-human animal, or another organism.

These instructions may refer to instructions customarily included in commercial packages of ELISA assay kits, immunohistochemistry (IHC) assay kits, immunofluorescence assay kits, flow cytometry assay kits, and immuno-colloidal gold assay kits. A kit of the present disclosure may also contain any other reagents useful for detecting the presence of pathogenic antibodies in an individual, such as 96-well microtiter plates, a non-specific protein such as bovine serum albumin, a secondary antibody that binds to an antibody of the present disclosure without affecting its antigen-binding, and reagents for detection, such as a fluorescent or luminescent label, or an enzyme and substrate that produce a detectable signal (e.g., horseradish peroxidase and TMB).

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Serious Adverse Reactions Caused by Anti-Coronavirus Antibodies

In our PCT/US2014/25918 (Biological therapeutics for infectious or inflammatory diseases or conditions), the pathogenic role of anti-influenza sera was disclosed using a timed-pregnant mouse model. The similar mouse model was used in the current application to evaluate the pathogenic role of anti-coronavirus antibodies and the therapeutic effect of the non-pathogenic antibodies for the prevention and treatment of the disorders caused by the pathogenic anti-coronavirus antibodies.

### Antibodies Specific for the Spike Protein of Coronavirus Caused Serious Adverse Reactions

The purified IgG of the rabbit anti-COVID-19, the anti-coronavirus antibodies used include rabbit polyclonal antibodies specific for the recombinant spike (S) or the nucleocapsid (N) proteins of the SARS-CoV-2 virus, and the recombinant spike proteins of SARS-CoV virus (Bioss Antibodies, Beijing); mouse monoclonal antibody specific for the recombinant nucleocapsid (N) proteins of the SARS-CoV virus; and naturally occurred human monoclonal antibodies specific for the receptor binding domain (RBD) of the spike protein (S-RBD) of the SARS-Co-V-2 virus, isolated from patients with the COVID-19 infection (provided by HuaAn McAb Biotech, Hangzhou, for research use only). The naturally occurred human monoclonal antibodies specific for the S-RBD of the COVID-19 (SARS-CoV-2) virus included antibodies of B38 (Wu et al, Science 368, 1274-1278; 2020), Regn10987 (Hansen et al., Science 369, 1010-1014; 2020), CC12.3 (Yuan et al., Science 369, 1119-1123; 2020), and Cr3022-b6 (bioRxiv preprint doi: https://doi.org/10.1101/2020.12.14. 422791).

Anti-(SARS-CoV-2) S, anti-COVID-19 (SARS-CoV-2) N, anti-SARS S1, anti-SARS N, human monoclonal anti-S-RBD antibodies of the COVID-19 (SARS-CoV-2) virus, B38 and Regn10987 as described above were used in the pregnant mouse model. The purified IgG of healthy rabbit, mouse and human as well as the human monoclonal anti-S-RBD antibodies of the COVID-19 (SARS-CoV-2) virus, Cr3022-b6, were used as controls. 50 μg (microgram) (about 2.0 mg/kg) and 60 μg (about 2.0 mg/kg) of each polyclonal antibody IgG and 40 μg (about 1.5 mg/kg), and 50 μg (about 1.5 mg/kg) of each monoclonal antibody IgG were injected intraperitoneally (IP) into timed-pregnant mice twice every three days at pregnancy day E15 (about 26-28 g) and E18 (about 30-32 g) respectively (FIG. 1A). The body weight of the pregnant mice was measured every day after the antibody injection. The mouse pups were born at about E20-E21 and the healthy status of the newborn mouse pups were observed and recorded. The course was ended at day 1 or 2 post birth. At the end day, the tissue samples of lung, heart, brain, kidney, liver and intestine from newborn mouse pups were collected for histological evaluation and immunofluorescent staining. The blood samples were collected from newborn mouse pups and the sera were isolated for cytokine detection.

Injection of the Regn10987 antibody into pregnant mice induced significant fetal death and neonatal death of the mouse pups delivered to the dames (p value: 0.02) (Table 1). The fetal death was confirmed by autopsy (FIG. 1B). The frequencies of the sick and death of the fetus and newborn mouse pups are summarized in FIG. 1C and Table 1. The results with this animal model indicated that the monoclonal antibody of Regn10987 is at the highest risk for inducing sick and death (61.9%), followed by the monoclonal antibody of B38 (45.8%) and the polyclonal anti-COVID-19 (SARS-CoV-2) S1 (45.5%). The polyclonal anti-SARS-CoV S also caused significant sick and death of the fetus and newborn mouse pups (37.6%). In addition, hyperemia at the end of left up and down limbs and a small hemangioma at the side of left eve of one pup was observed. The pup was delivered to a deme injected with the polyclonal anti-COVID-19 (SARS-CoV-2) S1 antibody. Neither the control antibodies nor the anti-COVID-19 N nor the anti-SARS-CoV N antibodies caused significant sick and death of the newborn mouse pups (Table 1).

TABLE 1
The sick and death rates of mouse newborns delivered to the
dames with the injection of anti-coronavirus antibodies

| Injected IgG of | N= | Sick (%) | Death (%) | Sick + Death (%) | Odds Ratio | 95% CI | P value |
|---|---|---|---|---|---|---|---|
| Saline | 17 | 0 | 5.88 | 5.88 | NA | NA | NA |
| Healthy rabbit serum | 6 | 0 | 16.7 | 16.7 | 3.20 | 0.17-61 | 1.00 |
| Healthy mouse serum | 7 | 0 | 11.1 | 11.1 | 2.67 | 0.14-50 | 1.00 |
| Healthy human serum pool* | 14 | 0 | 7.14 | 7.14 | 1.23 | 0.07-22 | 1.00 |
| Anti-COVID-19 N | 13 | 7.69 | 0 | 7.69 | 1.33 | 0.08-24 | 1.00 |
| Anti-COVID-19 S1 | 22 | 27.3 | 18.2 | 45.5 | 13.3 | 1.5-119 | 0.01 |
| Anti-COVID-19 S1 + N | 17 | 5.88 | 0 | 5.88 | 0.06 | 0.06-17 | 1.00 |
| Anti-SARS N | 15 | 6.67 | 13.3 | 20.0 | 4.00 | 0.37-43 | 0.30 |
| Anti-SARS S | 14 | 31.3 | 6.25 | 37.6 | 12.0 | 1.2-117 | 0.03 |
| MAb-Cr3022-b6 | 9 | 0 | 11.1 | 11.1 | 2.00 | 0.11-36 | 1.00 |
| MAb-B38 | 24 | 33.3 | 12.5 | 45.8 | 13.5 | 1.5-119 | 0.01 |
| MAb-Regn10987 | 35 | 17.1 | 44.8 | 61.9 | 21.3 | 2.5-179 | 0.006 |
| Regn10987 + 2MAbs** | 11 | 0 | 18.2 | 18.2 | 3.56 | 0.28-45 | 0.5 |

*Normal IgG pool of 4 healthy individuals without infection or vaccination of coronavirus
**MAb-Cr3022-b6 and MAb-CC12.3. Fisher Exact Probability Test, two tailed

### Histological Changes

The tissue sections of lungs, brains, hearts, kidneys, intestines and livers from the newborn mouse pups were stained with hematoxylin-eosin (HE) for histology evaluation. The human IgG or rabbit IgG bund on the tissues in vivo was detected by an immunofluorescent staining with fluorescent labeled secondary anti-human IgG or anti-rabbit IgG antibodies.

### Lung Inflammation and Injury

Acute lung inflammation was observed with the HE stained tissue sections from the mouse pups delivered to the dames injected with the anti-COVID-19 (SARS-CoV-2) S1, anti-SARS-CoV S (FIG. 2A), and the antibodies of Regn10987 and B38 (FIG. 2B). The lung lesion included pulmonary congestion, alveolar epithelial hyperplasia and thickening, alveolar atresia, alveolar dilatation and alveolar fusion. Infiltration of inflammatory cells and hemorrhage at the local lesion areas were observed.

### Other Organ Inflammation and Injury

Inflammatory reaction and hemorrhage were also observed with the tissues of kidney, brain and heart from the mouse pups as mentioned above.

The histology of the kidneys from the mouse pups delivered to the dames with the injection of anti-COVID-19 (SARS-CoV-2) S1, anti-SARS S, B38 and Regn10987 showed acute tubular necrosis (ATN). Renal tubular epithelial cells showed granular or vacuolar degeneration, dilated or obstructed lumen, and some of the epithelial cells fell off, renal interstitial edema with a small amount of inflammatory cells infiltration (FIG. 2C). Some mouse pup's kidney also showed glomerular endothelial cells proliferation, bleeding, glomerular necrosis and crescent body formation. The kidney injury caused by antibody Regn10987 was the most significant (FIG. 2C).

Small amount of cerebral or heart hemorrhage or inflammatory cells infiltration was observed with the brain and heart from a mouse pup delivered to a dame with injection of antibody Regn10987 (FIG. 2C). Large amount of inflammatory cells infiltration was observed with the brains and hearts from mouse pups delivered to the dames with injection of antibodies of SARS-CoV S, B38 and Regn10987 (data not shown).

Further, myocardial hemorrhage was observed with the hearts form the mouse pups delivered to the dames with injection of antibodies of anti-SARS S and B38. Myocardial swelling and inflammatory cells infiltration were observed with a mouse pup delivered to the dame with injection of antibodies of B38 (data not shown).

### In Vivo Antibody Binding to Multiple Diseased Tissues of Mouse Pups

As an evidence of the pathogenicity of the pathogenic antibodies, in vivo antibody binding to tissues of mouse pups was detected with an immunofluorescent staining as described above. The human and rabbit anti-COVID-19 (SARS-CoV-2) S antibodies were significantly detectable at the inflammatory and lesion areas of the tissues of lungs, kidneys, brains, hearts, livers and intestines from the mouse pups with server sickness (FIG. 3). Those mouse pups were delivered to the dames injected with the pathogenic antibodies of anti-COVID-19 (SARS-CoV-2) S1, anti-SARS S, Regn10987 and B38 (FIG. 3). Neither human IgG nor rabbit IgG was significantly detected on the tissues from the pups treated with the non-pathogenic antibodies of anti-COVID-19 (SARS-CoV-2) N, anti-SARS N, and the control antibody of Cr3022-b6. The results indicated that the anti-COVID-19 (SARS-CoV-2) spike antibodies went through the placenta, bound to the fetal tissues, mislead the self-attack immune responses, and induced systematic inflammation and injuries of multiple organs such as lungs, kidneys, and brains. The results are consistent to those of histological changes (FIG. 2) and provided the in vivo evidence for the pathogenicity of the anti-COVID-19 spike antibodies.

In summary, certain antibodies specific for the spike protein of the SARS-CoV-2 virus can be pathogenic and induce serious adverse reactions during the COVID-19 infection or the COVID-19 vaccination. The pathogenic antibodies can be induced during an infection (e.g. the COVID-19 or an influenza infection) or a vaccination (e.g. the COVID-19 or an influenza vaccination), or passively introduced (e.g. a therapeutic antibody). The diseases or conditions caused by pathogenic antibodies include infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, infection-relating autoimmune diseases, allergy and infection-relating cancers, and any other disorders (known or unknown) inducible by pathogenic antibodies. The diseases or conditions caused by pathogenic antibodies further include abortion, postpartum labor, still birth of pregnant females, and neonatal death and neonatal sudden death, caused by an infection or by a vaccine or by a pathogenic antibody.

### The Prevention and Treatment of the Adverse Reactions of Pathogenic Antibodies

It was surprising when the pathogenic anti-COVID-19 (SARS-CoV-2) S1 antibody was mixed with equal amount of the non-pathogenic anti-COVID-19 (SARS-CoV-2) N (50 μg+50 μg), the sick and death rate of the mouse pups delivered to the dames injected with the antibody mixture was significantly decreased compared to the mouse pups delivered to the dames injected with the anti-COVID-19 (SARS-CoV-2) S1 alone (Table 1). Moreover, the sick and death rate induced by the pathogenic Regn10987 antibody was also significantly decreased when a mixture of the antibody and other two non-pathogenic antibodies of Cr3022-b6 and CC12.3 was injected (Table 1). The mixture was consisted of 40 μg of Regn10987, 20 μg of Cr3022-b6 and 20 μg of CC12.3. It should be noted that the pathogenic action of the Regn10987 antibody was affected not through the competitive binding of the non-pathogenic antibodies since the biding sites of those antibodies are different. The data suggested that co-existing of non-pathogenic antibodies can reduce the pathogenicity of pathogenic antibodies. In another word, a vaccine capable of inducing non-pathogenic antibodies is safer. In yet another word, a vaccine capable of inducing multivalent antibodies is safer, in which at least one-valent antibody induces non-pathogenic antibodies that induce less adverse reactions in a host.

### Antibody Mixture Significantly Reduced Inflammatory Cytokine Production

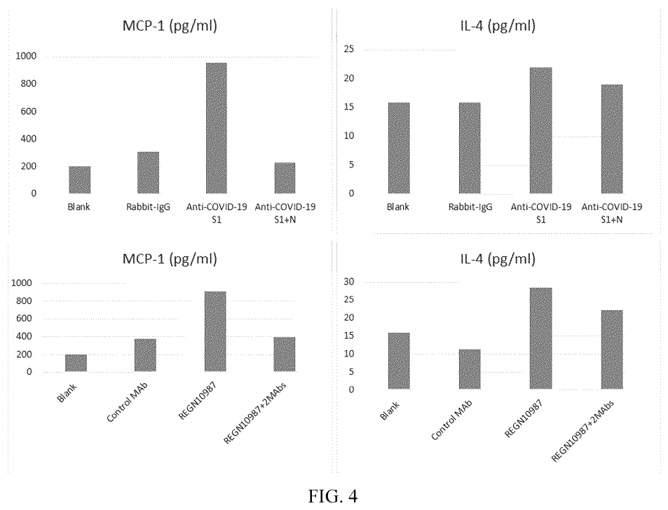

The sera from newborn pups were tested for inflammatory cytokines of MCP-1, TNF-α, IL-4, IL-6 and IL-10 using a 5-plex multiplex Luminex assay kit (Millipore) according to manufacturer's instruction. The results are summarized in FIG. 4. The antibodies of the anti-COVID-19 (SARS-CoV-2) S1 and the Regn10987 induced significantly higher levels of MCP-1 and IL-4 (FIG. 4). Consisted to the surprising histological results, the treatment of the antibody mixture comprised the pathogenic anti-COVID-19 (SARS-CoV-2) S1 and the non-pathogenic anti-COVID-19 (SARS-CoV-2) N, significantly reduced the cytokine level of MCP-1 (P<0.001) compared to the mouse pups delivered to the dames injected with the pathogenic anti-COVID-19 S1 alone. In addition, the treatment of the antibody mixture comprised the pathogenic Regn10987 and two of the non-pathogenic antibodies also significantly reduced the cytokine level of MCP-1 (P<0.001) compared to the mouse pups delivered to the dames injected with the pathogenic Regn10987 alone.

The levels of other cytokines were not significantly elevated probably due to the undeveloped immunity of the newborn mouse pups. The results were consistent with the results of the sick and death rates (Table 1) and the histology changes (FIG. 2). The data demonstrated that 1) pathogenic antibodies alone can induce high levels of inflammatory cytokines and have the potential to induce a cytokine storm or cytokine release syndrome (CRS); and 2) co-existing of non-pathogenic antibodies can reduce the inflammatory cytokine release induced by pathogenic antibodies and prevent the possible cytokine storm or CRS caused by the pathogenic antibodies.

Based on the results, a safer vaccine can be made by making the vaccine capable of inducing non-pathogenic antibodies. For example, a COVID-19 vaccine capable of inducing the antibodies specific for not only the spike protein but also for the nucleocapsid proteins or a non-spike protein of the SARS-CoV-2 virus is safer. For another example, a safer influenza vaccine capable of inducing the antibodies specific for not only the hemagglutinin (HA) protein but also the Neuraminidase (N) proteins or a non-HA protein of an influenza virus are safer. Those vaccines are better and safer because they induce less adverse reactions.

### Example 2: Pathogenic Antibodies Bind to Damaged Cells

Binding of anti-coronavirus and anti-influenza antibodies to healthy (intact) or damaged lung epithelium cells was tested with the human lung epithelium cell line A549. A549 cells were treated with sialidase according to manufacture's instruction (Roche) in order to induce damaged cells. The fluorescent labeled wheat germ agglutinin (WGA, Vector), which specifically binds to sialic acid, and a flow cytometry analysis were used to determine the levels of sialic acid on the surface of A549 cells. The damaged cells with missed sialic acid on cell surface were used to imitate the in vivo conditions of infected lung epithelium cells (sick cells).

The two human monoclonal antibodies specific for the COVID-19 (SARS-CoV-2) S-RBD protein, Regn10987 and B38, strongly bound to the damaged A549 cells with missed sialic acid (FIG. 5B). The Regn10987 also weakly bound to healthy A549 cells while the B38 did not bind to the healthy A549 cells (FIG. 5A). The control antibody of Cr3022-b6 bound to neither the healthy A549 cells nor the damaged cells (FIG. 5A). In addition, the antibodies specific for the spike glycoprotein of SARS-CoV virus (anti-SARS-CoV S) also strongly bound to the damaged A549 cells with missed sialic acid while neither of the antibodies bound to the healthy A549 cells with sialic acid (FIG. 5C). In addition, the polyclonal antibody specific for SARS-CoV-2 nucleocapsid protein (anti-COVID-19 N) and the antibody specific for SARS-CoV nucleocapsid protein (anti-SARS N) did not significantly bind to neither healthy nor damaged A549 cells (FIG. 5D). Furthermore, the antibodies of B38, Regn10987 and anti-SARS-CoV S strongly bound to the damaged human embryonic kidney HEK-293 cells with missed sialic acid. Neither the antibodies bound to the healthy HEK-293 cells. The antibodies of anti-SARS-CoV-2 N and the anti-SARS-CoV N did not bind to neither healthy nor damaged HEK-293 cells (data not shown).

Moreover, the anti-influenza viral antibodies of anti-H1N1 (California/09), anti-H3N2 and anti-B virus also significantly bound to the damaged A549 cells with missed sialic acid, compared to the healthy A549 cells (FIG. 5E). The results are consistent to the in vivo observations of the pathogenic actions of anti-influenza sera in a timed-pregnant mouse model, published in PCT/US2014/25918 (Biological therapeutics for infectious or inflammatory diseases or conditions).

Taken together, the results of the in vitro analysis provide a possible mechanism of action (MOA) of the pathogenic antibodies. The in vitro data indicated that certain antibodies specific for spike protein of the SARS-CoV-2 virus and the SARS-CoV virus have the potential to mislead the immune response to attack self by binding to the sick cells such as human lung epithelium cells or human embryonic kidney cells with damaged sugar chain on the cellular surface. This is consistent to the in vivo results as shown in Example 1. The Regn10987 antibody may have higher risk potential to activate immune responses since the antibody bind to not only the sick cells but also the heathy cells despite at a low rate. Similar pathogenic action was observed with the anti-influenza viral antibodies as well (probably related to the anti-HA antibodies), which is consistent to the in vivo observations published in PCT/US2014/25918 (Biological therapeutics for infectious or inflammatory diseases or conditions), in which the pathogenic role of anti-influenza sera was disclosed using a timed-pregnant mouse model.

### Example 3: Binding of Pathogenic Antibodies to Human Fetal or Diseased Tissues

In order to further evaluate the pathogenicity of the anti-COVID-19 (SARS-CoV-2) S antibody, the Regn10987 antibody with the highest pathogenic potential was tested for antibody binding to various human fetal tissues or multiple human diseased tissues from tissue array slides (US Biomax). The results are shown in FIGS. 6-7.

The Regn10987 antibody bound to the tested multiple human fetal tissues of lung, heart, kidney, brain, pancreas, liver, thymus and testicle (FIG. 6), indicating that the unmatured fetal tissues are vulnerable to a pathogenic antibody. In addition, the Regn10987 antibody bound broadly to the human inflammatory tissues or cancer tissues of respiratory, cardiovascular, urinary and digestive system (FIG. 7). The inflammatory tissues tested were from human pneumonia, bronchitis, bronchiectasis, valvular disease, rheumatoid valvular disease, myocarditis, esophagitis, gastritis, colitis, appendicitis, pancreatitis, and hepatitis. The cancer tissues tested were from human small cell lung carcinoma, kidney clear cell carcinoma, myxoma, etc. The data indicating that the most of actively proliferating cells or tissues such as the inflammatory tissues or some of cancer tissues are vulnerable to a pathogenic antibody such as the Regn10987.

### Example 4: Binding of Pathogenic Antibodies to Human Healthy Tissues

As further evidence of the pathogenicity of the pathogenic antibodies, the Regn10987 and multiple healthy human tissues from tissue array slides (US Biomax, FDA999i) were used to further evaluate the pathogenicity of the antibody. The tissue array slides comprised healthy human tissues of 33 of different organs.

The Regn10987 antibody bound to the tested human healthy tissues of lung, kidney, pancreas, stomach, intestine, adrenal gland, parathyroid gland, thyroid gland, spleen, adenohypophysis, testicle, prostate, bone marrow, uterine cervix of cancer adjacent normal tissue. The data indicating that some of anti-COVID-19 (SARS-CoV-2) S-RBD antibody such as the Regn10987 are highly pathogenic because it has the highly potential to induce serious reactions in vivo. Based on these results, detection of pathogenic antibodies during an infection at clinic is helpful to predict the consequences of a patient with a serious infection.

In summary, the pathogenic antibodies together with the damaged or actively proliferating cells or tissues such as the inflammatory cells or tissues can be the cause of serious infections preferably highly pathogenic viral infections (e.g. COVID-19 infection), serious adverse reactions of vaccines (e.g. COVID-19 vaccines) or pathogenic antibodies (e.g. anti-COVID-19 S antibodies), serious complications of infections (e.g. ARDS), infection-relating inflammation and autoimmune diseases, and infection-relating cancers which can occur if an inflammatory cellular proliferation stimulated by a pathogenic antibody repeatedly persists for long time and loses control. Further, the pathogenic antibodies can bind to the unmatured fetal cells or tissues and cause abortions, postpartum labors, still births of pregnant females, and neonatal deaths and neonatal sudden deaths. Therefore, the individual with pre-existing inflammatory diseases or injured tissues are vulnerable to an infection of highly pathogenic pathogens (e.g. COVID-19 infection), in which pathogenic antibodies can be induced by the pathogen. Furthermore, the individual with pre-existing inflammatory diseases or injured tissues are vulnerable to vaccine of highly pathogenic pathogens (e.g. COVID-19 vaccine), in which pathogenic antibodies can be induced by the vaccine of the highly pathogenic pathogen. It should be noted that the majority (70% or more) of the anti-COVID-19 (SARS-CoV-2) S antibodies inducible by either the COVID-19 (SARS-CoV-2) virus or a COVID-19 vaccine is non-pathogenic since the pathogenic antibodies take less than 30% according to the data of this disclosure.

The examples are considered to be sufficient to enable one skilled in the art to practice the invention. Other embodiments besides the above may be articulated as well. It is expected that others will perceive differences, which while differing from the foregoing, do not depart from the spirit and scope of the disclosure herein described and claimed.

1-94. (canceled)

95. A safer vaccine inducing less adverse reactions particular the serious adverse reactions, comprising at least one of:
1. a. a safer vaccine antigen of a pathogen that induce non-pathogenic antibodies to a host;
2. b. an isolated polynucleotide comprising a nucleic acid sequence encoding a safer vaccine antigen of a pathogen; and
3. c. a vector comprising a nucleic acid sequence encoding a safer vaccine antigen of a pathogen;
wherein the safer vaccine antigens of a pathogen induce non-pathogenic antibodies of the pathogen that cause less adverse reactions particular the serious adverse reactions in the host of the pathogen.

96. The safer vaccine of claim 95, wherein the safer vaccine antigens of a pathogen are an intact protein of a pathogen or fragments of a pathogen protein.

97. The safer vaccine of claim 95, wherein the isolated polynucleotide comprising a nucleic acid sequence encoding a safer vaccine antigen of a pathogen is DNA or mRNA, or is constructed in a vector.

98. The safer vaccines of claim 95, wherein the pathogens are selected from bacteria, viruses, fungi, viroids, and prions; preferably, the viruses are selected from the respiratory viruses, or the enteroviruses.

99. The safer vaccines of claim 98, wherein the respiratory viruses are selected from the coronaviruses, influenza viruses, respiratory enterovirus, adenovirus, rhinovirus, respiratory syncytial virus or B virus; preferably, the coronaviruses are selected from the SARS-CoV-2 virus, SARS-CoV viruses, MERS-CoV viruses; and preferably, the influenza viruses are selected from the type A, type B and type C influenza viruses; wherein the influenza A viruses are selected from at least one of H1N1, H3N2, H5N1, H7N9, H7N8 virus; wherein the enteroviruses are selected from rotavirus, reovirus, Coxsackie virus, Echoviruses, Enteroviruses, Polioviruses, norovirus, coronavirus, Norwalk virus, cytomegalovirus (CMV), herpes simplex virus, hepatitis virus, enteric cytopathic human orphan (ECHO) virus, porcine enterovirus (PEV), transmissible gastroenteritis virus (TGEV), foot and mouth disease (HFMD), human enterovirus 71, and porcine epidemic diarrhea virus (PEDV).

100. The safer vaccines of claim 95, wherein the safer vaccine antigens are selected from at least one of the surface proteins, the surface glycoproteins, the envelope proteins, the envelope glycoproteins, the membrane proteins, and the nucleocapsid proteins of a pathogen, wherein the selected proteins of the pathogen do not induce pathogenic antibodies in a host, wherein the pathogenic antibodies induce serious adverse reactions during an infection or a vaccination of the host; or the safer vaccine antigens are selected from at least one of the nucleocapsid proteins, the spike proteins, the spike glycoproteins, the envelope proteins, the envelope glycoproteins, the membrane proteins, the nucleocapsid proteins the glycans of the coronaviruses, wherein the coronaviruses are selected from SARS-CoV-2 virus, SARS-CoV viruses, MERS-CoV viruses; wherein the selected at least one of the proteins of the coronaviruses do not induce pathogenic antibodies in a host, wherein the pathogenic antibodies induce serious adverse reactions during an infection or a vaccination of the host; or the safer vaccine antigens are selected from at least one of the neuraminidase (NA) proteins, the hemagglutinin (HA) proteins, the other non-HA proteins, the envelope proteins, the envelope glycoproteins, the glycans, the capsid proteins and the nucleocapsid proteins of the influenza viruses; wherein the selected at least one of the proteins of the influenza viruses do not induce pathogenic antibodies in a host, wherein the pathogenic antibodies induce serious adverse reactions during an infection or a vaccination of the host; or the safer vaccine antigens are selected from at least two different antigenic epitopes of one pathogen protein, wherein at least one of the two different antigenic epitopes induces non-pathogenic antibodies; or the safer vaccine antigens are selected from at least two different proteins of a pathogen, wherein at least one of the two different proteins induces non-pathogenic antibodies.

101. The safer vaccines of claim 95, the safer vaccines induce monovalent antibodies, wherein the monovalent antibodies do not induce serious adverse reactions; or the safer vaccines induce multivalent antibodies, wherein at least one of the multivalent antibodies does not induce serious adverse reactions.

102. An isolated polynucleotide or a vector comprising a nucleic acid sequence encoding the safer vaccine antigens of claim 95.

103. An isolated host cell comprising the polynucleotide or the vector of claim 102.

104. A method of producing a safer vaccine antigen, comprising culturing the host cell of claim 103 that produces the safer vaccine antigen encoded by the nucleic acid, and recovering the vaccine antigen from the cell culture.

105. A safer vaccine antigen produced by the method of claim 104.

106. A composition comprising the safer vaccine antigens of claim 95, and a pharmaceutically acceptable carrier.

107. A method for treating or preventing infectious diseases, infection-relating diseases, and the adverse reactions particular the serious adverse reactions of vaccines or pathogenic antibodies in an individual, comprising administering to the individual an effective amount of a composition of claim 106, wherein the individual is a human or a non-human animal; wherein the composition comprising the safer vaccine antigens is administered intramuscularly, subcutaneously, orally, by implantation, by inhalation, intrathecally, or intranasally.

108. The method of claim 107, wherein the infectious diseases and the infection-relating diseases are caused by pathogens, wherein the pathogens are selected from bacteria, viruses, fungi, viroids, and prions; preferably, the viruses are selected from the respiratory viruses, or the enteroviruses; or the infectious diseases and the infection-relating diseases are caused by respiratory viruses, wherein the respiratory viruses are coronaviruses wherein the coronaviruses are selected from the SARS-CoV-2 viruses, the SARS-CoV viruses, and the MERS-CoV viruses; or the infectious diseases and the infection-relating diseases are caused by influenza viruses, wherein the influenza viruses are selected from the type A, type B and type C influenza viruses, wherein the influenza A viruses are selected from H1N1, H3N2, H5N1, H7N9, H7N8 virus; preferably, the infection-relating diseases are selected from the complications or sequela of infections, infection-relating autoimmune diseases, infection-relating allergies, infection-relating inflammation, and tumors that occurred during or after an infection.

109. The method of claim 107, wherein the adverse reactions particular the serious adverse reactions of vaccines are caused by the vaccines of the pathogens; or the adverse reactions particular the serious adverse reactions of vaccines are caused by the pathogenic antibodies inducible by the pathogens and the vaccines of the pathogens; or the serious adverse reactions of vaccines or pathogenic antibodies are selected from deaths, ARDS, coagulation abnormality, thrombocytopenia, stroke, blood clots, disseminated intravascular coagulation, Bell's palsy, COVID-19 long hauler, abortion, postpartum labor, still birth, neonatal death, acute infant death syndrome, cytokine storm, cytokine release syndrome, kidney failure, Guillain-Barre syndrome, Kawasaki's disease, acute leukemia, allergies, serious allergic reactions, severe nervous system reaction, life-threatening severe illness with multiple organ failure; wherein the pathogens are selected from bacteria, viruses, fungi, viroids, and prions; preferably, the viruses are selected from the respiratory viruses, or the enteroviruses.

110. A method for making safer vaccines comprising preparing a composition containing at least one of the safer vaccine antigens of a pathogen of claim 95, or containing at least one of the isolated polynucleotide or the vector comprising a nucleic acid sequence encoding the safer vaccine antigen of claim 95, and optional pharmaceutically acceptable adjuvants, carriers, excipients, or stabilizers.

111. A non-pathogenic antibody inducible by the safer vaccine antigens of claim 95, wherein the non-pathogenic antibodies are specific for the surface proteins, the surface glycoproteins, the envelope proteins, the envelope glycoproteins, and the membrane proteins of a pathogen, or the nucleocapsid proteins, the spike proteins, the spike glycoproteins, the envelope proteins, the envelope glycoproteins, the membrane proteins, the glycans and the saccharides of the coronaviruses, wherein the coronaviruses are SARS-CoV-2 virus, SARS-CoV viruses, MERS-CoV viruses; or the non-pathogenic antibodies are specific for the neuraminidase (NA) proteins, the non-HA proteins, the envelope proteins, the envelope glycoproteins, the capsid proteins and the nucleocapsid proteins of the influenza viruses, wherein the influenza viruses are type A, type B and type C influenza viruses, wherein the influenza A viruses are H1N1, H3N2, H5N1, H7N9, H7N8 virus.

112. A method for treating or preventing infectious diseases, infection-relating diseases, and the adverse reactions particular the serious reactions of vaccines or pathogenic antibodies in an individual, comprising administering to the individual an effective amount of a composition comprising at least one of the non-pathogenic antibodies of the claim 111; wherein the non-pathogenic antibodies are administered intramuscularly, intravenously, intra-articularlly, intracerobrospinally, by infusion, intraperitoneally, subcutaneously, intrasynovialy, intrathecally, orally, by inhalation, intranasally, and topically.

113. A method for detecting pathogenic antibodies in an individual, comprising:
(a) contacting a biological sample from the individual with the pathogenic antigens of any claim 112; and
(b) detecting binding of the antibody from the biological sample to the pathogenic antigens, wherein binding of the antibody from the biological sample to the pathogenic antigens may indicate the presence of pathogenic antibodies in the individual;
wherein the individual is a human or a non-human animal.

114. The method of claim 113, further comprising comparing the amount of antibody binding detected in step (b) with an amount of antibody binding to a control sample; preferably, the binding of the antibody from the biological sample to the pathogenic antigens is detected by an assay selected from the group consisting of an ELISA assay, a flow cytometry assay, an immunohistochemistry assay, an immunofluorescence assay, and an immune-colloidal gold assay; wherein the biological sample is selected from the group consisting of blood, serum, urine, feces, milk, semen, saliva, chest fluid, abdominal fluid, cerebrospinal fluid, sputum, and any other body fluid or secretion.

115. The method of claim 112, wherein the individual is infected with an infectious pathogen, wherein the pathogen is selected from bacteria, viruses, fungi, viroids, and prions; preferably, the viruses are selected from the respiratory viruses, or the enteroviruses; or the individual is infected with the coronaviruses, wherein the coronaviruses are SARS-CoV-2 virus, SARS-CoV viruses, and MERS-CoV virus; or the individual is infected with the influenza viruses, wherein the influenza viruses are type A, type B and type C influenza viruses, wherein the influenza A viruses are selected from H1N1, H3N2, H5N1, H7N9, H7N8 virus; wherein the individual is vaccinated with a vaccine relating to any one of the infectious pathogens, wherein the pathogens are selected from bacteria, viruses, fungi, viroids, and prions; preferably, the viruses are selected from the respiratory viruses, or the enteroviruses; or the individual is vaccinated with a vaccine of the coronaviruses, wherein the coronavirus vaccines are the vaccines of SARS-CoV-2 virus, SARS-CoV viruses, and MERS-CoV virus; or the individual is vaccinated with a vaccine of influenza viruses, wherein the vaccines are the vaccines of type A, type B and type C influenza viruses, wherein the vaccines of the influenza A viruses are selected from the vaccines of H1N1, H3N2, H5N1, H7N9, H7N8 virus.

116. A pathogenic antigen selected from a pathogen of claim 98, wherein the pathogenic antigen neutralizes the pathogenic antibodies inducible by the pathogen; wherein the pathogenic antigen is a fragment, a synthetic peptide, a glycan, a glycoprotein, a protein of pathogens of claim 98; or the pathogenic antigen is selected from the surface proteins, the surface glycoproteins, the envelope proteins, the envelope glycoproteins, and the membrane proteins of a pathogen, wherein the pathogenic antigen neutralizes the pathogenic antibodies inducible by the pathogen; or the pathogenic antigen is selected from the spike proteins, the spike glycoproteins, the envelope proteins, the envelope glycoproteins, the membrane proteins, the glycans of the coronaviruses, wherein the coronaviruses are selected from SARS-CoV-2 virus, SARS-CoV viruses, MERS-CoV viruses, wherein the pathogenic antigen neutralizes the pathogenic antibodies inducible during an infection or a vaccination of a coronavirus; or the pathogenic antigen is selected from the spike proteins, the spike glycoproteins, the spike 1 proteins, or the receptor binding domain (RBD) of spike proteins of the SARS-CoV-2 virus, or SARS-CoV viruses; or the pathogenic antigen is selected from the hemagglutinin (HA) proteins, the envelope proteins, the envelope glycoproteins, the glycans, and the capsid proteins of the influenza viruses, wherein the pathogenic antigen neutralizes the pathogenic antibodies inducible during an infection or a vaccination of an influenza virus.

117. A method for treating or preventing infectious diseases, infection-relating diseases, and the adverse reactions particular the serious reactions of vaccines or pathogenic antibodies in an individual, comprising administering to the individual an effective amount of a composition comprising at least one of the pathogenic antigens of the claim 116, wherein the individual is a human or a non-human animal; wherein the pathogenic antigens are administered intramuscularly, intravenously, intra-articularlly, intracerobrospinally, by infusion, intraperitoneally, subcutaneously, intrasynovialy, intrathecally, orally, by inhalation, intranasally, and topically; or the individual is infected with the coronaviruses, wherein the coronaviruses are SARS-CoV-2 virus, SARS-CoV viruses, and MERS-CoV virus; or the individual is infected with the influenza viruses, wherein the influenza viruses are type A, type B and type C influenza viruses, wherein the influenza A viruses are selected from H1N1, H3N2, H5N1, H7N9, H7N8 virus.
